(19) United States
(12) Patent Application Publication
(10) Pub. No.: US 2024/0233876 A1
(43) Pub. Date: Jul. 11, 2024

(54) METHODS FOR ENCODING INFORMATION IN THE PATTERN OF RNA TAILS ATTACHED TO A DOUBLE STRANDED DNA AND FOR READING SUCH INFORMATION USING SOLID STATE MEMBRANE NANOPORES

(71) Applicant: The Board of Trustees of the University of Illinois, Urbana, IL (US)

(72) Inventors: Jean-Pierre Leburton, Urbana, IL (US); Olgica Milenkovic, Urbana, IL (US); Rajat Chakraborty, Urbana, IL (US)

(73) Assignee: The Board of Trustees of the University of Illinois, Urbana, IL (US)

(21) Appl. No.: 18/402,438

(22) Filed: Jan. 2, 2024

Related U.S. Application Data
(60) Provisional application No. 63/438,242, filed on Jan. 10, 2023.

(51) Int. Cl. G16B 50/30; C12Q 1/6825; G01N 33/487

(52) U.S. Cl. CPC G16B 50/30; C12Q 1/6825; G01N 33/48721

(57) ABSTRACT

Systems and methods are provided for encoding information as the pattern of the lengths of RNA tails (or other tail biomolecules, e.g., polypeptides) attached to a dsDNA (or other backbone biomolecule) and the relative locations of attachment thereto. This information can be quickly and accurately read out by passing such information-encoding payload biomolecules through a nanopore and detecting the pattern of conductance of the pore (e.g., the ionic conductance through the pore and/or the transverse conductance of the membrane bearing the pore) as the payload passes therethrough. The conductance can be processed by subtracting a time-varying correction factor therefrom that represents the expected conductance for a ‘tail-free’ backbone as it transits through the pore, by applying a time-varying velocity factor that is dependent on the number of tails present in the pore over time to normalize individual tail dwell times, and/or other processing steps.

### CROSS-REFERENCE TO RELATED APPLICATION

This application claims priority to U.S. provisional application No. 63/438,242, filed Jan. 10, 2023, the contents of which are hereby incorporated by reference

### STATEMENT REGARDING FEDERALLY SPONSORED RESEARCH OR DEVELOPMENT

This invention was made with government support under contract numbers 1238993 and 1548562 awarded by the National Science Foundation. The government has certain rights in the invention.

### BACKGROUND

Genetics is a major field of research and of vital importance in life sciences and disease diagnosis. As current data storage technologies approach their density limits and require heavy maintenance costs, DNA data storage has been investigated. DNA data storage is predicted to have low energy and maintenance costs and approximately six orders of magnitude denser data density compared to current electronic and magnetic systems. However, storing data in DNA can include time-consuming laboratory processes for both synthesis and sequencing, making it impractical for widespread use. Some efforts have been deployed to store data in the sugar-phosphate backbone of double-stranded DNA (dsDNA) molecules instead of the bases e.g., by breaking one of the strands at various sites (nicks) on the backbone. In this approach, data is stored in the backbone of a dsDNA by using programmable restriction enzymes. This type of data storage also simplifies readout of the encoded information, because information of specific molecular characteristics can be detected instead of sequencing the whole DNA.

DNA sequencing has been dominated by Sanger's double-deoxygenation termination sequencing, this technology having reached a mature stage. This technique involves significant amounts of sample processing in the laboratory and is highly time-consuming. Alternatively, nanopore technologies have been investigated that include capturing DNA molecules and translocating them through a nanoscale pore. This label free technique involves decreased amounts of laboratory processing and is able to directly detect even certain types of epigenetic biomarkers. However, commercially available biological nanopores suffer from size restriction, lack of stability, and sensitivity to the experimental environment. Solid-state nanopores can address some of these drawbacks, and can provide the opportunity to tune the geometric shape and size of the nanopore for specific experiments and structures. These nanopores can provide better spatial resolution for detection as two-dimensional (2D) solid-state materials (e.g., graphene and molybdenum disulfide (MoS₂)) are characterized by thicknesses in the range of 0.3 nm to 0.7 nm, comparable to the separation between two consecutive base-pairs (bp) of a double-stranded DNA (dsDNA). Solid-state nanopores open up the possibility for large scale detection using innovative techniques e.g., multipore and multilayer devices. In these structures, biomolecule detection is achieved via in-plane conductance together with ionic current blocking. In recent years, transition metal dichalocogenide (TMD) e.g., MoS₂ membranes have gained significant attention because of their long-term stability and low hydrophobicity compared to graphene. These membranes have been successfully fabricated and studied both theoretically and experimentally, showing that their in-plane conductance detection provides better resolution for modified DNA structures compared to ionic currents.

### SUMMARY

A robust and reliable detection scheme of RNA tails grown on a double-stranded DNA (or some other tail biomolecule attached to a backbone biomolecule) is provided herein that can provide higher density information encoding than, e.g., DNA-based storage systems that use the “punch card” mechanism of encoding information in nicks along a DNA backbone. The conductance of a pore (e.g., a transverse conductance across an MoS₂ or other membrane that includes the pore, an ionic conductance through the pore) as such a payload biomolecule traverses through the pore can be detected and used to estimate the lengths and relative locations of the tails along the backbone, allowing the information encoded therein to be quickly, accurately, and cost-effectively read out. Algorithmic approaches are also provided herein to process such conductance signals to detect the presence of the RNA tails (or other tail biomolecules) on the double-stranded DNA (or other backbone biomolecule) as well as to differentiate among the tail lengths from the conductance signal (e.g., from the transverse conductance of MoS₂ membrane nanopores). All-atom molecular dynamics simulations with electronic transport modeling were used to validate these methods, showing that they can be used to detect the relative locations and lengths of RNA tails with lengths of 10, 15, and 20 nucleotides separated by 10 base-pairs along a backbone dsDNA. These methods can be extended to greater numbers of possible tail lengths, alternative sets of tail lengths, and alternative tail biomolecule and backbone biomolecule compositions. Dwell times can be normalized (e.g., using normalized DNA velocities that depend on the number of overlapping tail biomolecules that are simultaneously in the pore) to provide an improved method to detect and distinguish the lengths of the tail biomolecules.

Without wishing to be bound by any particular theory, there can be discussion herein of beliefs or understandings of underlying principles or mechanisms relating to embodiments of the disclosure. It is recognized that regardless of the ultimate correctness of any explanation or hypothesis, an embodiment of the disclosure can nonetheless be operative and useful.

The foregoing and other objects and features of the disclosure will become more apparent from the following detailed description, which proceeds with reference to the accompanying figures.

Further embodiments, forms, features, aspects, benefits, objects, and advantages of the present application shall become apparent from the detailed description and figures provided herewith.

In a first aspect, a method is provided that includes: (i) applying a voltage, a concentration gradient, or a mechanical pressure to a solution that contains a payload, wherein the payload comprises a backbone biomolecule with a plurality of tail biomolecules attached thereto at respective different locations along the backbone biomolecule, wherein the solution is divided into first and second volumes separated by a barrier, and wherein the barrier has a pore such that applying the voltage, concentration gradient, or mechanical pressure to the solution causes the payload to move from the first volume to the second volume through the pore; (ii) while the payload moves from the first volume to the second volume through the pore, measuring a time-varying conductance of the pore, wherein measuring the time-varying conductance of the pore comprises at least one of measuring a time-varying ionic conductance through the pore or measuring a time-varying transverse electronic conductance of the barrier; and (iii) based on the time-varying conductance of the pore, determining lengths of the plurality of tail biomolecules and the relative locations of attachment of the plurality of tail biomolecules to the backbone biomolecule.

In a second aspect, a method is provided that includes: (i) generating, on a backbone biomolecule, a plurality of nicks, and (ii) forming, at each nick of the plurality of nicks, a respective tail biomolecule, wherein the lengths and relative locations along the backbone biomolecule of the tail biomolecules encode payload information.

In a third aspect, a system is provided that includes: (i) a barrier having a pore; and (ii) a controller comprising one or more processors, wherein the controller is configured to perform controller operations comprising: (a) applying a voltage, a concentration gradient, or a mechanical pressure to a solution that contains a payload, wherein the payload comprises a backbone biomolecule with a plurality of tail biomolecules attached thereto at respective different locations along the backbone biomolecule, wherein the solution is divided into first and second volumes by the barrier, and wherein applying the voltage, concentration gradient, or mechanical pressure to the solution causes the payload to move from the first volume to the second volume through the pore; (b) while the payload moves from the first volume to the second volume through the pore, measuring a time-varying conductance of the pore, wherein measuring the time-varying conductance of the pore comprises at least one of measuring a time-varying ionic conductance through the pore or measuring a time-varying transverse electronic conductance of the barrier; and (c) based on the time-varying conductance of the pore, determining lengths of the plurality of tail biomolecules and the relative locations of attachment of the plurality of tail biomolecules to the backbone biomolecule.

In a fourth aspect, a non-transitory computer readable medium is provided having stored thereon program instructions executable by at least one processor to cause the at least one processor to perform the method of the first or second aspect.

In a fifth aspect, system is provided that includes: (i) a controller comprising one or more processor, and (ii) a non-transitory computer readable medium having stored thereon program instructions executable by the controller to cause the controller to perform the method of the first or second aspect.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are included to provide a further understanding of the system and methods of the disclosure and are incorporated in and constitute a part of this specification. The drawings illustrate one or more embodiment(s) of the disclosure, and together with the description serve to explain the principles and operation of the disclosure.

FIG. 1A depicts aspects of a payload, according to example embodiments.

FIG. 1B depicts the payload of FIG. 1A moving through a pore in a barrier, according to example embodiments.

FIG. 1C depicts an example time-varying correction factor.

FIG. 1D depicts aspects of determining the relative locations and lengths of RNA tails attached to the payload of FIG. 1A, according to example embodiments.

FIG. 2A depicts aspects of a simulated experimental setup including an example payload and an example barrier having a pore, according to example embodiments.

FIG. 2B depicts simulated experimental results.

FIG. 3 depicts simulated experimental results.

FIG. 4 depicts simulated experimental results.

FIG. 5 depicts simulated experimental results.

FIG. 6 depicts simulated experimental results.

FIG. 7 depicts aspects of a simulated experimental setup, according to example embodiments.

FIG. 8 depicts simulated experimental results.

FIG. 9 depicts simulated experimental results.

FIG. 10 depicts aspects of a simulated experimental setup, according to example embodiments.

FIG. 11 depicts aspects of a simulated experimental setup, according to example embodiments.

FIG. 12 depicts simulated experimental results.

FIG. 13 depicts simulated experimental results.

FIG. 14 depicts aspects of a simulated experimental setup, according to example embodiments.

FIG. 15 depicts aspects of an example system.

FIG. 16 depicts aspects of an example method.

FIG. 17 depicts aspects of an example method.

### DETAILED DESCRIPTION

The following detailed description describes various features and functions of the disclosed embodiments with reference to the accompanying figures. The illustrative embodiments described herein are not meant to be limiting. It may be readily understood that certain aspects of the disclosed embodiments can be arranged and combined in a wide variety of different configurations, all of which are contemplated herein.

### I. OVERVIEW

Long-term data storage is important, with various different technologies (e.g., magnetic tape) having different benefits and drawbacks with respect to cost, throughput, density, stability, and other factors. The use of DNA or other complex biomolecules to store data shows promise for increased storage density and long-term data retention. However, it is currently difficult, slow, and expensive to encode information into the pattern of base pairs of a DNA molecule (e.g., via oligonucleotide synthesis) and to read out such information once stored. For example, various Sanger sequencing-based Next Generation Sequencing (NGS) methods can retrieve such information, but in a slow and expensive process that implicates significant laboratory equipment, reagents, and operator time. In another example, nanopores can be used to read out the sequence of the DNA as a time-varying pattern of pore conductance as the DNA passes through the pore; however, the magnitude of the conductance signals involved is very low, and their relationship to the pattern of base pairs is very complex, making accurate recovery of the information difficult.

Instead of encoding information in the pattern of amino acids, base pairs, nucleotides, or other ‘sequence’ aspects of a DNA, RNA, protein, or other biomolecule, the embodiments described herein encode information into a payload biomolecule via a pattern of the lengths and locations of attachment of tail biomolecules (e.g., RNA tails, polypeptide tails) to a backbone biomolecule (e.g., a double-stranded DNA molecule). As the backbone biomolecule of such a payload biomolecule passes through a pore (e.g., a nanopore formed in an MoS₂ membrane), the tail biomolecules attached thereto are ‘dragged along’ through the pore. This causes the pattern of the number of biomolecule tails present in the pore over time being based on the pattern of lengths and locations of attachment of those tails to the backbone biomolecule. Thus, this pattern of presence and number of tails in the pore over time can be detected and used to “read out” the information encoded in a payload biomolecule.

The presence or absence of a tail biomolecule in the pore causes a significant change in the electronic properties of the pore (e.g., an ionic conductance through the pore, a transverse electronic conductance across an MoS₂ or other membrane that includes the pore) due to the correspondingly large change in the number of atoms in or near the pore when such a biomolecule tail is or is not present. These electronic changes are much larger than the changes observed relating to the identity of the nucleotides of a DNA or RNA strand as it passes through such a pore, or to the identity of amino acids of a polypeptide as it passes through such a pore. Thus, the lengths and location of attachment of such biomolecule tails can be significantly more easily read out electronically than the sequence of a single biomolecule. This makes the present method of encoding information in the pattern of lengths and locations of attachment of tail biomolecules to a backbone biomolecule an attractive option for long-term, high-density information storage in biomolecules.

Such a payload biomolecule can be created to encode payload information in a variety of ways. For example, where the backbone biomolecule is a double strand of DNA (“dsDNA”), the locations on the backbone biomolecule of a subset of the tail biomolecules having the same length could be nicked. The same-length subset of the tail biomolecules could then be grown from the nick sites and/or attached already fully or partially grown to the nick sites. This could be done for each of the discrete lengths of tail biomolecules to be attached to the backbone molecule separately, to allow the lengths of each subset to differ. Each length of biomolecule could be grown completely separately. For example, RNA tails could be grown from a set of nick sites on a backbone DNA and then terminated. Alternatively, the growth could occur in an overlapping manner. For example, the sites of the longest RNA tails could be nicked, and then the longest RNA tails grown for a set length (e.g., 5 nucleotides). The sites of the second-longest RNA tails could then be nicked, and then the longest RNA tails and the second-longest tails grown for an additional set length (e.g., 5 nucleotides, resulting in the longest tails being 10 nt long and the second-longest 5 nt long). This process could be repeated, in order of tail length, until the shortest tails are grown, resulting in tails of each length having been grown, cumulatively across multiple growth phases, to their respective different lengths.

The use of multiple (e.g., 2, 3, or more) different lengths of RNA tails (or other biomolecule tails) attached to specified sites on a backbone biomolecule (e.g., dsDNA), together with the ability to efficiently and accurately read out the relative locations and lengths of such tails on the backbone, leads to a paradigm shift in biomolecule-based data storage due to the ability of storing more than log₂(2)=1 bit of information per nicking site. Whereas merely nicking sites using positional encoding of data results in the ability to signal with only the presence (bit 1) and absence (bit 0) of nicks, the storage of information in specified-length biomolecule tails can significantly increase the information density of such payload biomolecules.

For example, specified-length RNA tails can be grown at the locations of nicks in the sugar-phosphate backbone of DNA by, e.g., removing a phosphodiester bond and a phosphate group at the 5′ end of a DNA strand using Pyrococcus furiousus Argonaute (PfAgo) or Streptococcus pyogenes Cas9 nickage (SpCas9n) and/or targeting specific sites to create a nick using DNA methyltransferase (M. TaqI). RNA tails can then be attached to the nicks by, e.g., using oligodeoxynucleotides (AdoYnODN11). The specified, differing lengths of the RNA tails can be obtained by attaching already-grown tails of different lengths in sequential nicking and attachment steps, or by growing the tails in situ at the various attachment sites for respective different periods of time (in an overlapping or non-overlapping manner, as described above).

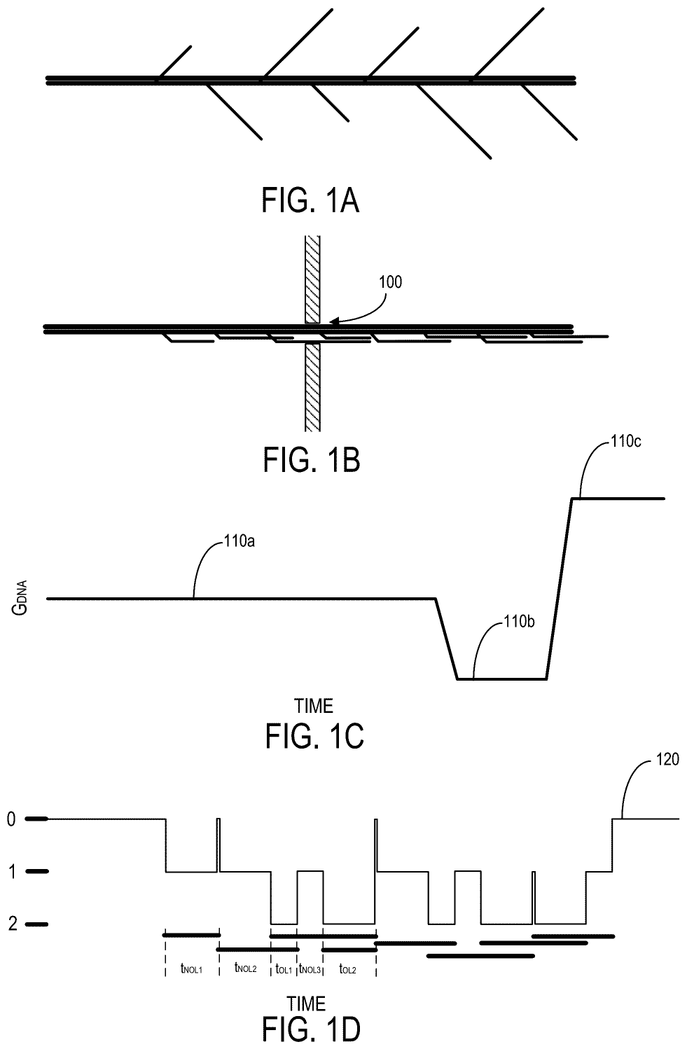

FIG. 1A depicts schematically a payload biomolecule as describe herein, which includes a backbone biomolecule (depicted as the horizontal double-line) to which is attached a plurality of tail biomolecules (thinner single lines at angles). The lengths and relative location of attachment of the tail biomolecules to the backbone biomolecule can be specified to encode some payload information that can be read out from the payload biomolecule by measuring the lengths and relative locations of attachment of the tail biomolecules to the backbone biomolecule (e.g., using the read out methods described herein).

The encoding scheme for mapping payload information to the lengths and locations of attachment of the tail biomolecules could take a variety of forms. For example, the locations of attachment could be regularly spaced and the bits of the payload information encoded, n bits at a time, in the length of the tail biomolecules at each attachment site. For the payload biomolecule of FIG. 1A, whose tail biomolecules take one of three different lengths, the tail length could be mapped lengthwise to two bits, no tail to longest tail encoding 00 to 11, with the payload biomolecule encoding 01 10 11 01 10 11 11 10. Other encodings are possible, e.g., where the sites of attachment are not regularly spaced and so can, themselves, represent some additional payload information.

Aspects of the synthesis or read-out of the payload biomolecules could be constrained in some manner that restricts the configuration of the lengths and attachment locations of the tails from being independent. For example, the pattern of lengths and locations of attachment of the tails could be constrained such that the lengths and locations of the tails can be unambiguously read out from measured pore conductance(s) via a relatively simple algorithm. The information encoding method could take into account such constraints while maximizing the ability of the payload biomolecules, within the constraints, to encode such information. In some examples, the encoding method could include some redundancy to account for damage to the payload biomolecule and/or errors in the synthesis of the payload, errors in read-out of the payload, or other factors related to specific methods for “writing” or “reading” information to or from a payload biomolecule as described herein.

To “read” the information encoded in a payload biomolecule, the payload biomolecule can be passed through a pore in a barrier (e.g., a nanopore formed through a sheet of MoS₂) and, while the payload is passing through the pore, measuring one or more electronic properties of the pore that are related to the number of the tail biomolecules that are in the pore over time with the backbone biomolecule. As the backbone biomolecule passes through the pore, the tail biomolecules attached thereto will also be pulled through the pore and aligned with the backbone biomolecule such that the pattern of the number of tails in the pore over time can be used to infer the pattern of the lengths and relative locations of attachment of the tails to the backbone. FIG. 1B illustrates the passage of the payload biomolecule of FIG. 1A through a pore 100 formed through a barrier; as the backbone biomolecule passes through the pore, the tail biomolecules are aligned therewith in order to also pass through the pore 100.

Thus, the measured electronic propert(ies) of the pore can be used to determine the pattern of lengths and locations of attachment of the tails to the backbone, and thus to read out the payload information encoded in the payload biomolecule. The detected electronic propert(ies) of the pore could include an ionic conductivity through the pore and/or a transverse conductivity of the barrier that includes the pore. The payload biomolecule could be induced to pass through the pore in a variety of ways. For example, a voltage could be applied to the solutions on either side of the pore, leading to a voltage gradient through the pore that acts to drive a backbone biomolecule therethrough. In another example, a higher pressure could be mechanically induced on one side of the barrier (e.g., by exerting force onto a fluid-filled cylinder that is in fluidic communication with the solution on one side of the barrier) in order to drive the backbone biomolecule through the pore. In yet another example, a concentration gradient of one or more chemical species (e.g., ions) between one side of the pore and the other could act to drive the backbone biomolecule through the pore. Additional or alternative methods, or combinations of methods, could be used to induce the backbone biomolecule of a payload biomolecule as described herein to pass through a pore. The measured electrical properties of such a pore (e.g., transverse conductivity of the barrier that includes the pore, an ionic conductance through the pore) could be used to determine the lengths of the plurality of tail biomolecules and the relative locations of attachment of the plurality of tail biomolecules to the backbone biomolecule in a variety of ways. For example, a Markov chain, trained machine learning model, or other statistical method could be used to determine the patterns of lengths and locations of attachment from one or more measured electronic properties of the pore, or even to determine the payload information from such information directly without the intermediate determination of the pattern of lengths and locations of attachment. In some examples, the measured time-varying conductance of the pore can be translated into a time-varying number of the plurality of tail biomolecules moving through the pore simultaneously as the backbone biomolecule transits through the pore (pulling the tails attached thereto through the pore aligned with the backbone). This time-varying pattern of the number of tails in the pore can then be used to determine the lengths of the tails and their relative locations of attachment to the backbone biomolecule.

Such a time-varying pattern of the number of tails in the pore can be determined from a time-varying conductance of the pore in a variety of ways. For example, a baseline time-varying correction factor, related to the expected conductance of the pore if there were no tail biomolecules in the pore, could be subtracted off of the time-varying conductance of the pore prior to further analysis. Such a time-varying correction factor could be dependent on the length of the backbone biomolecule and could vary over time as the conductance of a ‘bare’ backbone biomolecule, having no tail biomolecules attached thereto, might be ‘expected’ to. Thus, the ‘remainder’ conductance, following the subtraction of such a correction factor, could be related more to the number of tails in the pore over time and less to the ‘baseline’ conductance of the backbone biomolecule.

Such a time-varying correction factor could take a variety of forms. Generally, as a backbone dsDNA strand passes through a pore, it exhibits a number of ‘regimes’ of conductance. During an initial phase, the dsDNA ‘straightens out,’ reducing the number of atoms in the vicinity of the pore (relative to a hypothetical, more-‘coiled’ dsDNA in or near the pore) and leading to a first-period conductivity for the time-varying correction factor. As the dsDNA nears the end of its transit through the pore, the remaining portion of the DNA on the ‘upstream’ side of the pore may coil up (e.g., as this terminal portion of the DNA does not experience drag from further-upstream portions of the backbone). This results in a second-period conductivity for the time-varying correction factor that is less than the first-period conductivity, as this ‘coiling’ causes more atoms of the backbone to be present in the vicinity of the pore. Finally, as the DNA backbone fully leaves the pore, the conductivity increases to the open-pore conductivity (since there are no longer any backbone atoms in the vicinity to reduce the conductivity of the pore), resulting in a third-period conductivity for the time-varying correction factor that is greater than the first-period conductivity.

A time-varying correction factor that reflects this pattern could be modeled as, e.g., a piecewise linear function, with constant portions having conductances that correspond to these first-, second-, and third-period conductances. FIG. 1C depicts an example of such a time-varying correction factor, which includes an initial constant segment 110a having the first-period conductance, a second constant segment 110b having the second-period conductance, and a terminal constant segment 110c having the third-period conductance. The particular values of the various conductances, the slopes and duration of the non-constant segments, and the duration of the second constant segment 110b could be determined based on calibration data measured for a particular pore or pore-containing apparatus, e.g., by passing a number of tail-free payload biomolecules and/or payload biomolecules of known configuration through a pore and measuring the time-varying conductance(s) of the pore as such biomolecules transit therethrough.

The time-varying conductance of the pore could also be normalized in order to facilitate determining therefrom the time-varying pattern of the number of tails in the pore. This could include, e.g., normalizing the time-varying conductance of the pore to the first-period conductance of time-varying correction factor after subtracting the time-varying correction factor from the time-varying conductance of the pore. Such a normalized time-varying conductance could then be compared to one or more thresholds in order to determine the time-varying pattern of the number of tails in the pore. FIG. 1D depicts an example of such a time-varying pattern of the number of tails in the pore 120 as the payload biomolecule passes through the pore 100. Such a time-varying pattern 120 could be determined, e.g., by subtracting the time-varying correction factor of FIG. 1C from a measured time-varying conductance of the pore 100, normalizing the difference by the first-period conductance of the time-varying correction factor, and then comparing that normalized signal to two threshold values, the first threshold value distinguishing “no tails present in the pore” from “a single tail present in the pore,” and the second threshold value distinguishing “a single present in the pore” from “two tails present in the pore.”

Note that the example payload biomolecule is configured such that no more than two tails are ever in the pore at the same time. Thus, the time-varying pattern of the number of tails in the pore 120 never exceeds 2. However, a payload biomolecule as described herein and methods for encoding and/or reading payload information to/from such a payload biomolecules may include greater numbers of tail biomolecules passing simultaneously through a pore.

Such a time-varying pattern of the number of tails in the pore 120, regardless of the method employed to generate it, could then be used to determine the lengths and relative locations of tail biomolecules attached to the backbone biomolecule of the payload biomolecule. Such a method of determination could proceed based on knowledge about the space of configurations that were used to generate the payload biomolecule. For example, it may be known that the payload biomolecule is configured such that, while passing through the pore 100, no more than two of the tail biomolecules will also be present in the pore 100 at the same time and that the maximum length of the tails is less than twice the separation between neighboring tails (or, alternatively, that the separation between neighboring tails is more than half the maximum length of the tails). In such an example, the pattern of lengths and relative locations of attachment of the tails can be unambiguously determined from the time-varying pattern of the number of tails in the pore 120. Such a pattern, determined from the time-varying pattern of the number of tails in the pore 120, is depicted in FIG. 1D as the set of horizontal lines. The length of each line depicts the length of a corresponding tail of the payload, while the left end of each line depicts the relative location of attachment along the backbone biomolecule of the corresponding tail of the payload. Alternatively, the spacing of attachments of the tails to the backbone could be specified based on the lengths of the tails to avoid ambiguities and/or to avoid more than a maximum number of the tails being in the pore at the same time.

As noted above, the payload biomolecule can be configured such that the time-varying pattern of the number of tails in the pore measured while passing the payload through the pore allows the pattern of lengths and relative locations of attachment of the tails to be unambiguously determined. For example, the payload could be configured such that, while passing through the pore 100, no more than two of the tail biomolecules will be present in the pore 100 at the same time and further such that the separation between the attachment locations of neighboring tails along the backbone is more than half of a maximum length of the tails. In such an example, a simple state machine can operate along the time-varying pattern of the number of tails in the pore to determine the start and end of each tail biomolecule when aligned to the backbone biomolecule. For example, for the time-varying pattern of the number of tails in the pore 120, this could include ‘starting’ a tail with any positive transition of the signal from ‘0’ to ‘1’ or from ‘1’ to ‘2,’ and ‘ending’ a tail (the ‘longer’ or ‘older’ tail, if there is an option between two already-existing tails) with any negative transition of the signal from ‘1’ to ‘0’ or from ‘2’ to ‘1.’ Such a method could be expanded to greater numbers of possible tails simultaneously present in a pore.

As the number of tails in the pore increases, the velocity of translation of the payload biomolecule through the pore can decrease, with greater decreases in velocity for greater numbers of tails simultaneously passing through the pore. Thus, it can be beneficial to normalize the observed duration over which a particular tail transited a pore by an amount related to the number of tails in the pore at the same time as the particular tail and to the duration(s) of overlap therewith. The normalized duration could then be used to determine the length of the particular tail and/or its location of attachment relative to other tails and, thus to decode the payload information represented by the payload biomolecule. This could include determining a sum of (i) duration that a particular tail was located in a pore without overlapping with any other tail, normalized by a first ‘non-overlapping’ velocity factor, and (ii) durations of time that the particular tail was located in the pore with one, two, or more other tails, each duration normalized by a respective ‘single tail-overlapping,’ ‘two tails-overlapping,’ or other velocity factor(s) for additional numbers of tails overlapping in the pore.

So, for example, the normalized duration of time for the first tail of FIG. 1D could be determined as (νₙₒₗ/ν_{dna})*t_{NOL1}; the normalized duration of time for the second tail of FIG. 1D could be determined as (νₙₒₗ/ν_{dna})*t_{NOL2}+(νₒₗ/ν_{dna})*(t_{OL1}); the normalized duration of time for the third tail of FIG. 1D could be determined as (νₙₒₗ/ν_{dna})*t_{NOL3}+(νₒₗ/ν_{dna})*(t_{OL1}+t_{OL2}); and the normalized duration of time for the fourth tail of FIG. 1D could be determined as (νₒₗ/ν_{dna})*(t_{OL2}). In these normalizations, ν_{dna} represents the velocity of the backbone DNA through the pore without any RNA tails also moving through the pore, νₙₒₗ represents the velocity of the backbone DNA through the pore with a single, non-overlapping RNA tail also moving through the pore, and νₒₗ represents the velocity of the backbone DNA through the pore with two overlapping RNA tails also moving through the pore. Where payloads are involved that exhibit more than two tails in the pore simultaneously, additional velocity correction factors could be used.

The singular terms “a,” “an,” and “the” include plural referents unless context clearly indicates otherwise. Similarly, the word “or” is intended to include “and” unless the context clearly indicates otherwise. “Comprising” means “including”; hence, “comprising A or B” means “including A” or “including B” or “including A and B.” All references cited herein are incorporated by reference.

The disclosure may be further understood by the following non-limiting examples. All references cited herein are hereby incorporated by reference to the extent not inconsistent with the disclosure herewith. Although the description herein contains many specificities, these should not be construed as limiting the scope of the disclosure but as merely providing illustrations of some of the presently preferred embodiments of the disclosure. For example, thus the scope of the disclosure should be determined by the appended aspects and their equivalents, rather than by the examples given.

While the present disclosure can take many different forms, for the purpose of promoting an understanding of the principles of the disclosure, references are made throughout to the embodiments illustrated in the drawings and specific language is used to describe the same. It will nevertheless be understood that no limitation of the scope of the disclosure is thereby intended. Any alterations and further modifications of the described embodiments, and any further applications of the principles of the disclosure as described herein are contemplated as would normally occur to one skilled in the art to which the disclosure relates.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present disclosure has been specifically disclosed by specific exemplary embodiments and optional features, modification and variation of the concepts herein disclosed may be resorted to by those skilled in the art, and that such modifications and variations are considered to be within the scope of this disclosure as defined by the appended aspects. The specific embodiments provided herein are examples of useful embodiments of the present disclosure and it will be apparent to one skilled in the art that the present disclosure may be carried out using a large number of variations of the devices, device components, methods steps set forth in the present description. As will be obvious to one of skill in the art, methods and devices useful for the present methods can include a large number of optional composition and processing elements and steps.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this disclosure for a given application. In specific applications, the selection of a given anion or cation for preparation of a salt may result in increased or decreased solubility of that salt.

Every formulation or combination of components described or exemplified herein can be used to practice the disclosure, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising,” “consisting essentially of,” and “consisting of” may be replaced with either of the other two terms. The disclosure illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the disclosure without resort to undue experimentation. All art-known functional equivalents, of any such materials and methods are intended to be included in this disclosure. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended aspects.

Although the present disclosure has been described with reference to certain embodiments thereof, other embodiments are possible without departing from the present disclosure. The spirit and scope of the appended aspects should not be limited, therefore, to the description of the preferred embodiments contained herein. All embodiments that come within the meaning of the aspects, either literally or by equivalence, are intended to be embraced therein. Furthermore, the advantages described herein are not necessarily the only advantages of the disclosure, and it is not necessarily expected that all of the described advantages will be achieved with every embodiment of the disclosure

### Definitions

A coding sequence is the part of a gene or cDNA which codes for the amino acid sequence of a protein, or for a functional RNA such as a tRNA or rRNA.

Complement or complementary sequence means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′.

Downstream refers to a relative position in DNA or RNA and is the region towards the 3′ end of a strand.

Expression refers to the transcription of a gene into structural RNA (rRNA, RNA) or messenger RNA (mRNA) and subsequent translation of an mRNA into a protein.

A nucleic acid construct is a nucleic acid molecule which is isolated from a naturally occurring gene or which has been modified to contain segments of nucleic acids which are combined and juxtaposed in a manner which would not otherwise exist in nature.

Nucleic acid molecule means a single- or double-stranded linear polynucleotide containing either deoxyribonucleotides or ribonucleotides that are linked by 3′-5′-phosphodiester bonds.

A polypeptide is a linear polymer of amino acids that are linked by peptide bonds. Upstream means on the 5′ side of any site in DNA or RNA.

### II. EXPERIMENTAL RESULTS

Methods to identify RNA tails attached to the backbone of a strand of double-stranded DNA (dsDNA) by signal processing analysis of transverse electric conductance variations along a MoS₂ nanopore membrane were experimentally investigated via simulation. By extracting the DNA+RNA tails dwell time from differential transconductance signals, the evaluated approach was able to detect the presence of RNA tails. Various lengths of the tails were also detectable by computationally adjusting the individual tail dwell times via a normalized DNA velocity technique. The methods described herein were validated by all-atom molecular dynamics (MD) and electronic transport modeling that represented the dynamics of the RNA dwell time variations resulting from different separations between RNA tails. An assessment of the robustness of these techniques across different substrate DNA lengths and numbers of RNA tails was also performed.

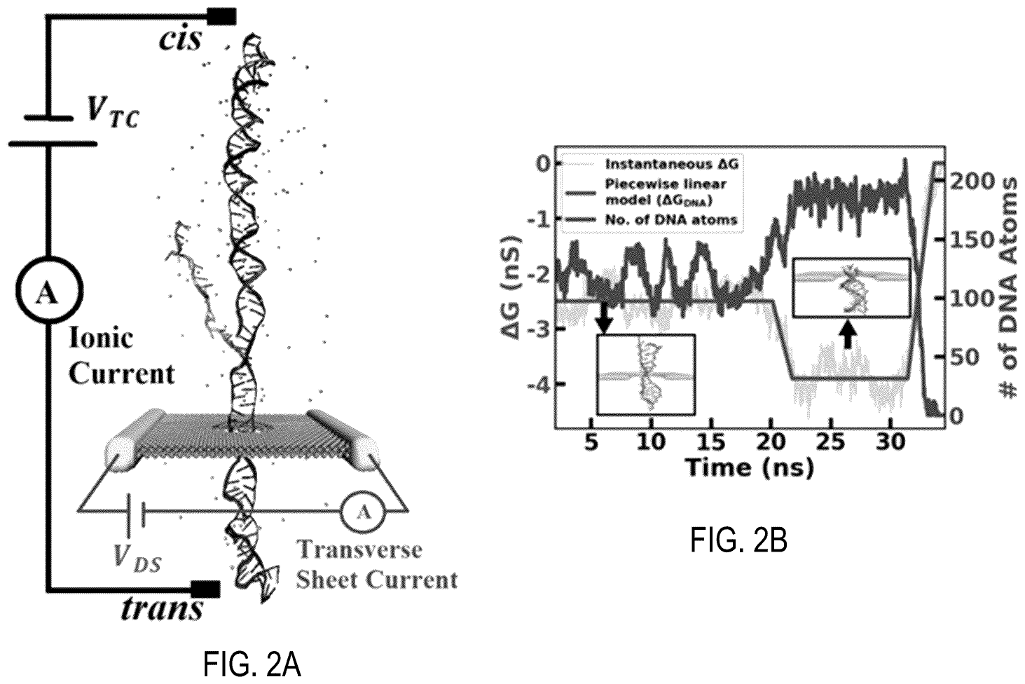

FIG. 2A illustrates aspects of an experimental setup for the detection of RNA tails attached to (e.g., grown on) a dsDNA, which was simulated by MD and electron transport modeling. In this set up, an electrically active 2D MoS₂ membrane separates an electrolytic cell into cis- and trans-chambers. A voltage, V_{TC} was applied along the direction perpendicular to the membrane plane, which generated an ionic current through the nanopore as well as translocated DNA molecules from the cis chamber to the trans chamber. A transverse electric field (driven by a voltage V_{DS}) was applied along the MoS₂ membrane in order to detect variations in the transverse electronic conductance of the membrane during the DNA translocations.

In the computational experimental approach, a 70 bp long dsDNA having a random sequence and with RNA tails of various lengths attached thereto (collectively, a ‘payload’) was immersed in a neutral ionic solution of 100 mM KCL. An end of this payload biomolecule was already present inside a 3-nm diameter nanopore in a 9 nm×9 nm MoS₂ membrane at the start of simulation, with 3-4 bp of the dsDNA already through the nanopore. This pore size was chosen to restrict the lateral movement of the DNA during translocation. A detailed description of the simulation is included below.

FIG. 2B displays both the number of DNA atoms in the vicinity of the pore and the differential conductance (ΔG) over simulated time, obtained by subtracting the open pore conductance (Gₒₚₑₙ) from the conductance (G) obtained during the translocation of a ‘pristine’ (i.e., having no RNA tails attached thereto) dsDNA through the nanopore, i.e., ΔG=G_{DNA}−Gₒₚₑₙ<0. Three distinct regimes of this time-varying measurement are observable in the figure. The first regime is an initial regime ranging from 0 ns to ˜20 ns, during which the number of DNA atoms in the vicinity of the pore (defined for purposes of illustration as within a distance from 5 A° below to 5 A° above the membrane) oscillated around 250, due at least in part to the DNA stretching and other conformational changes occurring during the initial phase of the translocation. The instantaneous ΔG also maintained a stable value with small fluctuations (˜0.5 nS) during this period that corresponded to these conformational changes and deformation due to the DNA interaction with the pore rim, which is depicted in the left inset. The second regime ranged from ˜22 ns to ˜32 ns, when the number of DNA atoms near the pore increased significantly (˜375), but with smaller fluctuations compared to the previous regime. Here, the DNA molecule was in the last phase of its translocation with few of its atoms left above the membrane. It recovered its usual helicoidal shape (right inset) when its interaction with the pore rim was reduced by the absence of its atoms above the membrane. The conformational changes then reduced as the DNA filled the space in the pore, and consequently increased the number of atoms around the pore. This behavior lowered the instantaneous ΔG in this regime. Finally, the terminal third regime was beyond ˜34 ns, when the DNA had completely left the pore, and both the number of atoms dropped to zero and the instantaneous ΔG vanished. This stereotypical instantaneous ΔG signal, representing the ‘typical’ ΔG that would be observed if DNA in the pore had no RNA tails attached thereto (e.g., if the DNA in the pore was ‘pristine’), was modeled as a piecewise linear function (ΔG_{DNA}) of time with two distinct horizontal levels which were labeled as ‘deformation’ (ΔG_{DNA-def}) during the first regime and ‘helicoidal shape’ (ΔG_{DNA-hel}) during the second regime. The final regime, after the DNA had passed completely from the pore, was modeled as ΔG=0.

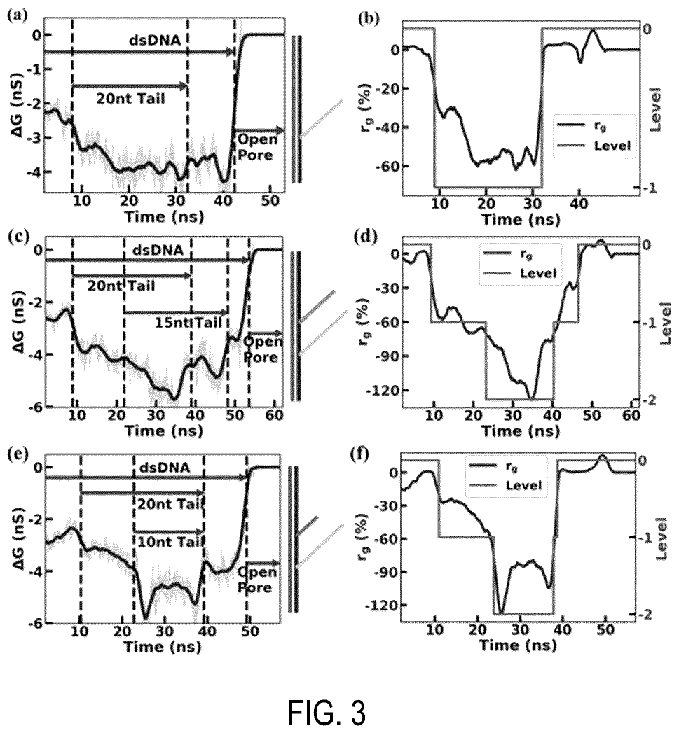

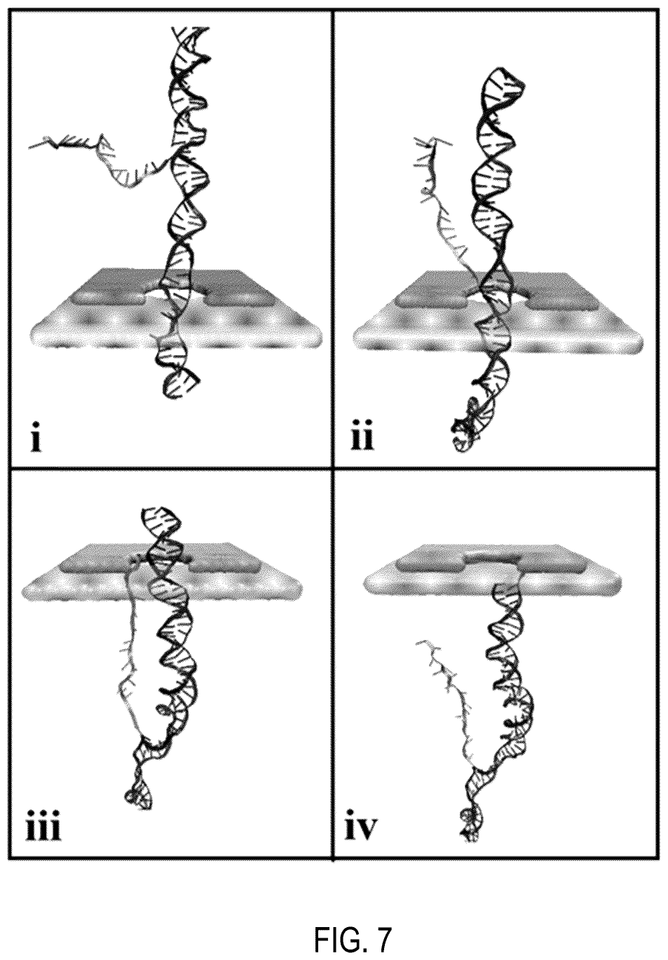

FIG. 3 depicts aspects of a process to identify the existence of RNA tails attached to a dsDNA and to extract their dwell times from the differential conductance signals for different configurations of such a payload biomolecule. FIG. 3(a) displays the differential conductance (ΔG) signal caused by the translocation of a 70 bp dsDNA with a single 20 nt RNA tail attached at the 28th base, through a 3 nm-diameter nanopore, as a function of time. The dsDNA that was present inside the pore at the start of simulation exited the pore after ˜43 ns while the attached tail entered the pore at ˜8 ns and exited at ˜32 ns. These events were obtained from MD, and the durations and boundaries are represented by red arrows and dotted vertical lines, respectively (the snapshots for these events are also shown in FIG. 7). Although there was a distinguishable drop in ΔG when the tail entered the pore (˜ 8 ns), there was no rise when the tail left the pore as seen from FIG. 3 (a). The lower differential conductance due to the presence of the RNA tail in the pore coincided with the drop due to the resumption of the helicoidal shape of the DNA near the end of translocation (the “second regime” as discussed in connection with FIG. 2B), which offset the expected rise in the raw signal due to the tail exiting the pore. In order to obtain the contribution of the RNA tail in the signal alone, a relative conductance, r_{g}, is defined as follows:

$${r}_{g} = \frac{Δ G - Δ {G}_{DNA}}{\vert Δ {G}_{DNA - def} \vert} \times 100 \% \qquad ( 1 )$$

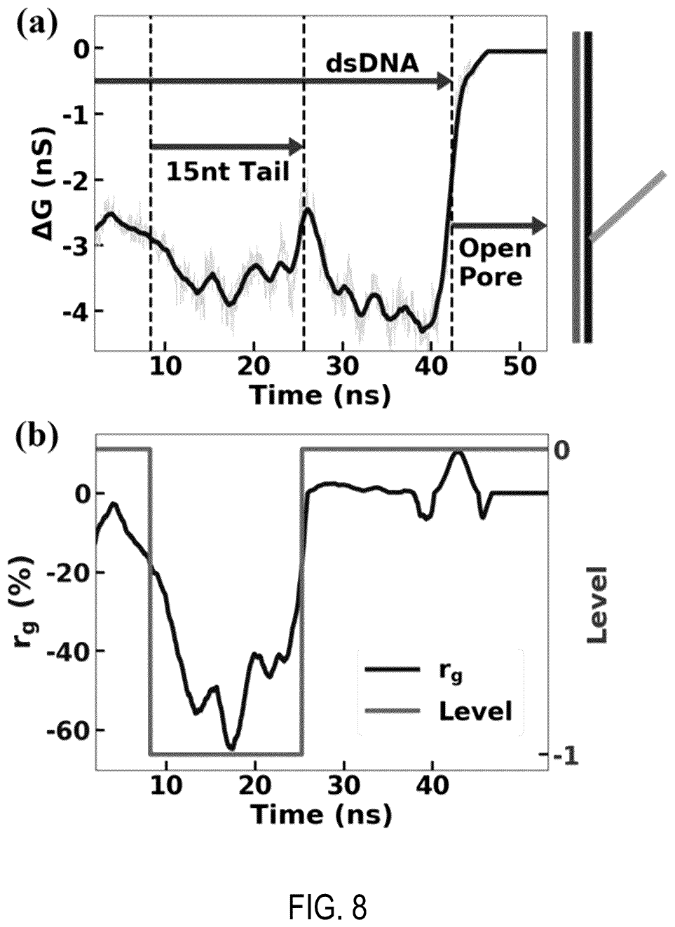

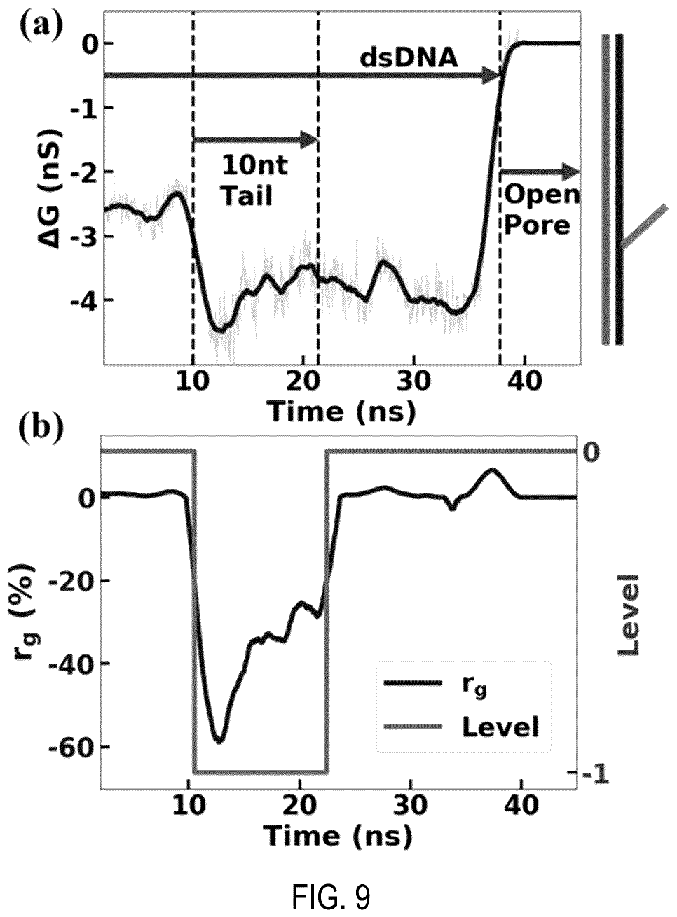

Here, ΔG=G_{DNA+RNA}−Gₒₚₑₙ<0 is the measured differential transverse conductance trace for the dsDNA, including the RNA tails, relative to the open pore conductance; ΔG_{DNA} is the ‘pristine’ or ‘baseline’ time-varying differential conductance trace for a dsDNA to which no RNA tails are attached (which may be implemented as the piecewise linear model described above), and ΔG_{DNA-def} is the differential conductance value of deformed DNA without RNA tails attached thereto during the first phase of translocation (e.g., the constant value of the piecewise linear trace during the initial regime, as described in FIG. 2B). FIG. 3(b) shows a plot of the relative conductance, r_{g}, for the DNA molecule with the 20 nt RNA tail as a function of time. A fall and a rise in the r_{g} signal can be clearly observed at ˜8 ns and ˜32 ns, respectively, when the DNA contribution is removed from the trace via the methods described above (e.g., by subtracting a piecewise linear model of the expected conductance of a tail-free DNA). A relative conductance threshold value (L₁) was then selected, below which the presence of an RNA tail is detected in the signal. In the present case, L₁=−20% was selected, which defined a “level −1,” while the absence of any tail (and thus the signal being higher than L₁) being denoted by “level 0,” as seen in FIG. 3(b). The duration that the signal occupied level −1 was the dwell time of the RNA tail in the nanopore, which for the 20 nt RNA tail was 23.1 ns. This is in good agreement with the dwell time obtained from MD snapshots (23.8 ns). The dwell times for 15 nt and 10 nt RNA tails attached at the same position as the 20 nt tail were calculated to be 17.1 ns and 11.94 ns, respectively. Differential and relative conductance signals for these tails are shown in FIG. 8 and FIG. 9.

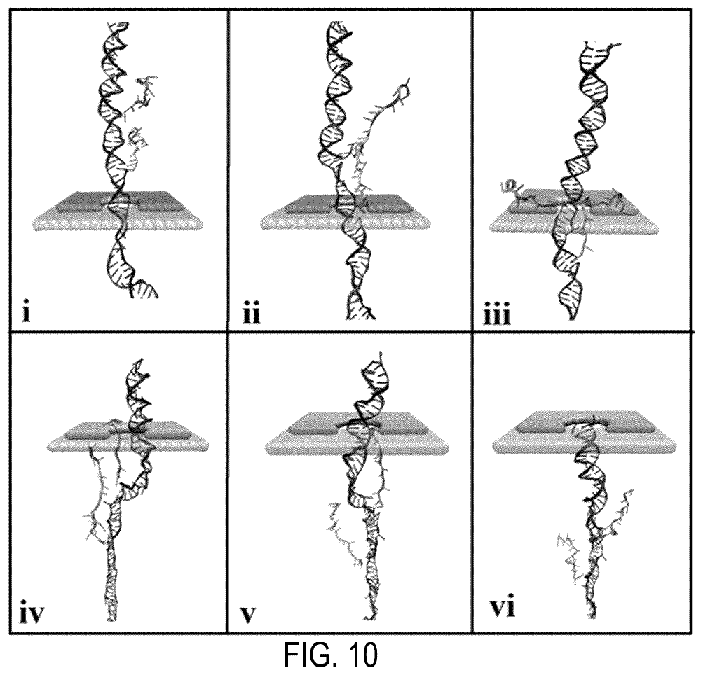

Next, the case of 20 nt and a 15 nt RNA tails placed 10 bp apart (at 28th and 38th base of the 70 bp dsDNA) was investigated. The resulting signals are shown in FIG. 3(c) and FIG. 3(d). The differential conductance trace (ΔG) in FIG. 3(c) indicates that the dsDNA, which began the simulation inside the pore, exited it at ˜54 ns. The 20 nt tail entered the pore at ˜9 ns and left at ˜39 ns, while the 15 nt tail entered at ˜23 ns and left at ˜47 ns. As seen from FIG. 3(c), the 20 nt tail signal overlaps with the 15 nt RNA tail signal between ˜23 ns and ˜39 ns because their separation (10 bp) is shorter than the length of the first (20 nt) tail, as the overlapping took place for a fraction of their overall dwell times. These events were obtained from MD simulation as in the previous scenario (boundaries and durations are shown by dotted vertical lines and red arrows, respectively, in FIG. 3(c)) and the corresponding snapshots are shown in FIG. 10. From Equ. (1), the first tail (20 nt) was detected when r_{g} fell below L₁, which was assign as “level −1” in FIG. 3(d). Next, the existence of the 15 nt tail (overlapping with the 20 nt tail) can be detected when r_{g} falls below a second threshold, L₂ (selected as −70%), which is denoted by “level −2” in FIG. 3(d). When r_{g} subsequently increases over L₂, it indicates that the 20 nt tail has left the pore to return to level −1. Finally, r_{g} rises above L₁ indicating that all tails have left the pore (level 0). The duration of the different events (two non-overlapping and one overlapping) was then calculated from their corresponding levels in FIG. 3(d) to derive the total dwell time for these two tails, 31.14 ns and 23.43 ns for the 20 nt and 15 nt tails, respectively, in agreement with MD simulations.

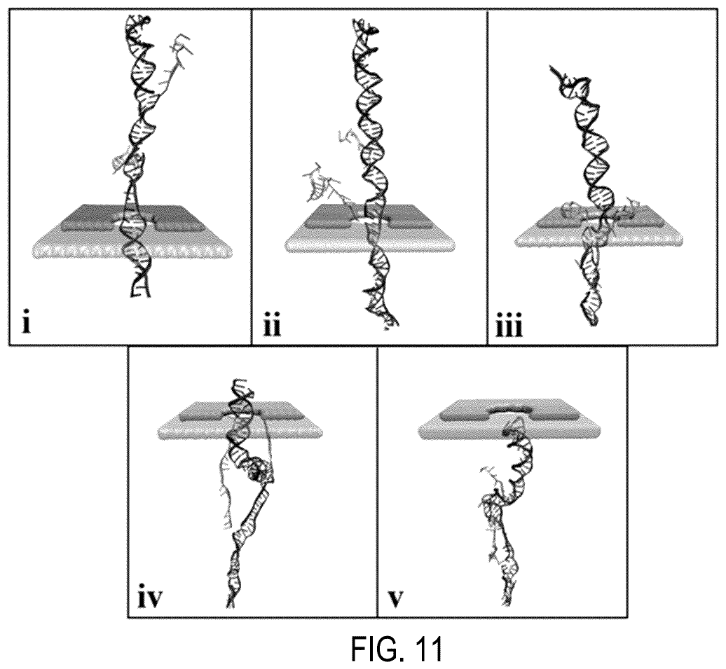

FIG. 3(e) and FIG. 3(f) show the signal of a first RNA tail of 20 nt completely overlapping with a second RNA tail of 10 nt placed 10 bp further along the backbone at the 28th and 38th base of the 70 bp dsDNA, respectively. The differential transconductance signal (ΔG) and events (obtained from MD) in FIG. 3(e) shows that the DNA (located inside the pore at the onset of simulation) exited the pore at ˜49 ns, the 20 nt tail entered the pore at ˜10 ns, then the 10 nt tail entered at ˜23 ns and later both tails left at ˜38 ns. The ΔG trace of FIG. 3(e) and the snapshots in FIG. 11 confirm that the 10 nt tail overlapped with the 20 nt tail for the entirety of the former's (10 nt tail) dwell time. The corresponding calculated relative conductance, r_{g} and various signal levels established by the techniques described herein are displayed in FIG. 3(f). FIG. 3(f) illustrates that the techniques described herein can successfully detect the existence of both RNA tails, and also extract their dwell times, which for the 10 nt tail and 20 nt tail were 14.97 ns and 26.88 ns, respectively. All the dwell times discussed above are also tabulated in Table 1 (a).

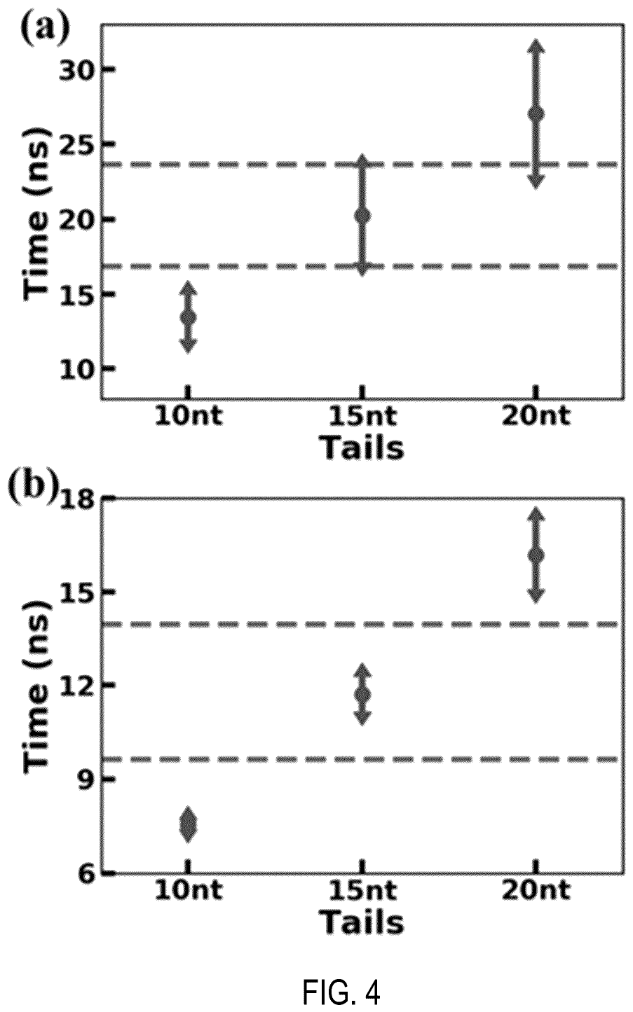

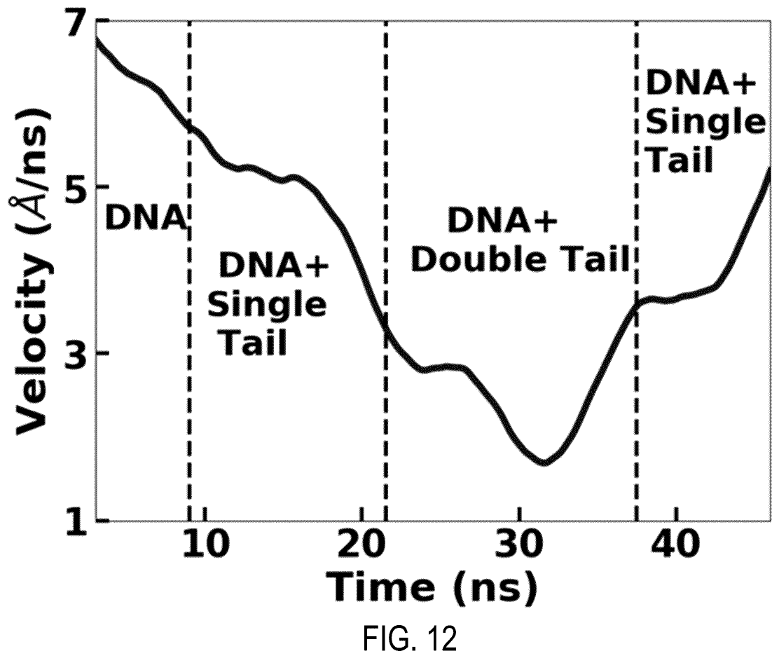

The length of the RNA tails of a payload as described herein can be determined based on the dwell times of the RNA tails. FIG. 4(a) shows the resulting dispersion (error bar) for the non-normalized dwell times of different tail lengths as obtained from Table 1 (a). Dashed green lines in FIG. 4(a) indicate the separation between the population of dwell times for two different tails obtained by bisecting the regions between the centers of the error bars. In FIG. 4(a), three distinct regions for the different tail lengths were identified (10 nt, 15 nt and 20 nt) based on these lines. Such separation lines may be referred to as “decision boundaries,” as the length of an unknown tail can be determined by considering which region (defined by such decision boundaries) contains the observed tail dwell time. The non-normalized dwell times of the 15 bp tail region overlap with those of both 10 bp tail and 20 bp tail, while the non-normalized dwell times of the 20 bp tail extend well into the region of 15 bp tail, even for this small number of sample points. Table 1 (a) clearly shows a significant increase in dwell times whenever there is an overlap of tails (i.e., whenever more than one RNA tails is transiting through the pore at the same time). This is related to the decrease in DNA translocation velocity perpendicular to the nanopore membrane during such overlapping-tail circumstances. An example of a simulated velocity profile for a 20 nt and a 15 nt tail placed 10 bp apart on a 70 bp dsDNA (this payload was also discussed in relation to FIG. 4) is shown in FIG. 12 with different stages of translocation. As seen from this figure, the velocity of the biomolecule exceeded ˜5.6 A°/ns when only the DNA (and no RNA tails attached thereto) was present inside the pore. When a single RNA tail was present in the pore along with the DNA, the velocity varied from ˜3.2 A°/ns to ˜5.6 A°/ns, and in the presence of two overlapping RNA tails, the velocity fell below ˜3.2 A°/ns. As the number of RNA tails increases, the area for translocation inside the pore shrinks and the interaction between the biomolecule and the pore rim increases. These effects result in a reduction of the biomolecule velocity, which in turn increases the detected tail dwell times of the tails. Accordingly, detected dwell times can be normalized to account for changes in velocity for such different events (e.g., the presence of one or more RNA tails in the pore, the number of RNA tails simultaneously within the pore). Such normalization can facilitate distinguishing the length of the RNA tails attached to the DNA of a payload biomolecule as described herein. An example calculation for a normalized dwell time (tₙₒᵣₘ) in scenarios were the expected payload is not likely to result in more than two RNA tails in the pore simultaneously is:

$${t}_{norm} = \frac{{v}_{ol}}{{v}_{dna}} {t}_{ol} + \frac{{v}_{nol}}{{v}_{dna}} {t}_{nol} \qquad ( 2 )$$

Here, vₙₒₗ and vₒₗ are the velocities during translocation of the dsDNA with non-overlapping RNA tails (i.e., only one RNA tail) and overlapping RNA tails (i.e., two RNA tails) inside the pore, respectively. v_{dna} is the DNA velocity when only the DNA (and no RNA tails) is inside the pore. tₙₒₗ and tₒₗ are the observed durations of time during which a particular RNA tail resides inside the pore in non-overlapping and overlapping states, respectively. Depending on the configuration of the payload biomolecule (e.g., the particular lengths and relative spacing of the RNA tails attached to the DNA strand), one or the other of these observed durations may represent the sum of the durations of two (or more) separate periods of time. v_{dna}, vₙₒₗ and vₒₗ were calculated by averaging the biomolecule velocity at all instants for the different respective scenarios from MD data; values of 6.1 nA°/ns, 3.98 nA°/ns, and 2.91 nA°/ns were obtained. The dwell times obtained by this renormalization procedure are tabulated in Table 1 (b) with the new error bar plots, generated based on the normalized dwell times for the observed population of RNA tails, displayed in FIG. 4(b). The plot shows that the normalized dwell times were more focused at the center of their respective regions compared to the original, non-normalized dwell times.

This methodology results in more well-defined criteria, in terms of the normalized dwell time, for characterizing the observed lengths of the tails. tₙₒᵣₘ<9.64 ns, 9.64 ns<tₙₒᵣₘ<13.95 ns and tₙₒᵣₘ>13.95 ns were determined as the dwell time threshold criteria to distinguish 10 nt, 15 nt, and 20 nt tails, respectively, based on the normalized dwell time. This process can be extended to payloads that, when read by passing through a pore as described herein, exhibit more than two RNA tails simultaneously passing through the pore. This extension can include obtaining decision criteria separating each adjacent number of overlapping tails, e.g., based on observed or simulated biomolecule velocities through the pore for the various numbers of overlapping tails in the pore.

To allow for simplified recovery of the tail dwell times for two-or-fewer overlapping RNA tail payloads, the minimum distance between two tails of the payload along the DNA can be maintained at more than or equal to half of the length of the longest tail. This can be done to ensure that more than two tails are never present in the pore at the same time, allowing the beginning and end of each tail to be unambiguously determined from the measured conductance trace via straightforward and computationally inexpensive methods.

The length differences between the two (or more) possible RNA tail lengths of a data-encoding payload biomolecule as described herein can be large enough that the decision boundaries of the normalized dwell times allow for the populations of RNA tails of different lengths to be unambiguously distinguishable from each another in the space of the normalized dwell time. The mean dwell times and velocities of multiple known payload samples could be experimentally measured for a particular experimental setup (e.g., a particular configuration of apparatus, a particular example of a pore or other element(s) of an apparatus) to enhance the reliability of the decision boundaries. For large numbers of samples, clustering methods (e.g., k-means clustering) can be used to determine the decision boundaries.

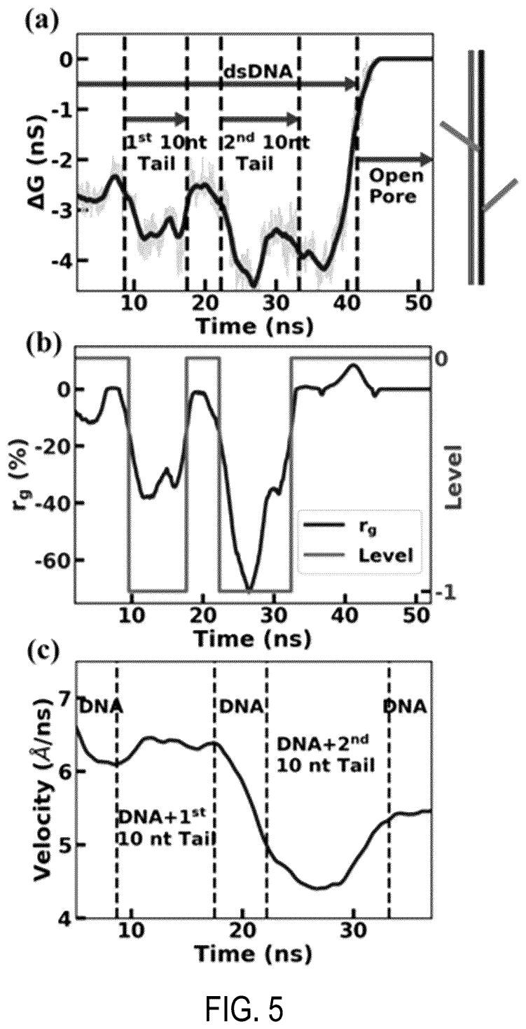

In FIG. 5, the tail length detection technique described herein is assessed when two tails with lengths of 10 nt are placed at 28^{th} and 43^{rd} base of the 70 bp dsDNA (15 bp separation). The differential transconductance signal in FIG. 5(a) reflects that the first tail entered the pore at ˜9 ns and exited at ˜18 ns, while the second tail entered the pore at ˜22 ns and left the pore at ˜32 ns. The plots for the relative conductance and levels were obtained by using the techniques described above. FIG. 5(b) shows that the original dwell times for these two tails were found to be 8.1 ns and 10.11 ns, respectively. The first tail had a smaller dwell time compared to the scenarios of FIG. 5 and FIG. 9. These differences are related to the velocity profile of the translocation, shown in FIG. 5(c). The translocation velocity of the first tail through the nanopore was much higher than vₙₒₗ (more than ˜6 A°/ns), thereby reducing dwell time. Since a single twist of the helical DNA structure has a length of about 10 bp, a separation of 15 bp (one and a half twist) puts the tails opposite each other on the double-helical structure. The electric field along the vertical direction then exerts a uniform force on both sides of the biomolecule, favoring the translocation of the tail through the nanopore (compared to the scenarios discussed in FIG. 5 and FIG. 9), resulting a higher velocity. Accordingly, the longer tails had similar reductions in the dwell time and similar overlap with respect to the decision region of the shorter tails. These factors indicate that scenarios wherein RNA tails are separated by

$$n + \frac{1}{2}$$

twists (n being zero or a positive integer) implicate more complex analysis. It can also be beneficial to attach the final tail significantly far away (more than the length of the longest RNA tail) from the end of the dsDNA to avoid finishing translocation of the dsDNA before the final tail has completely translocated.

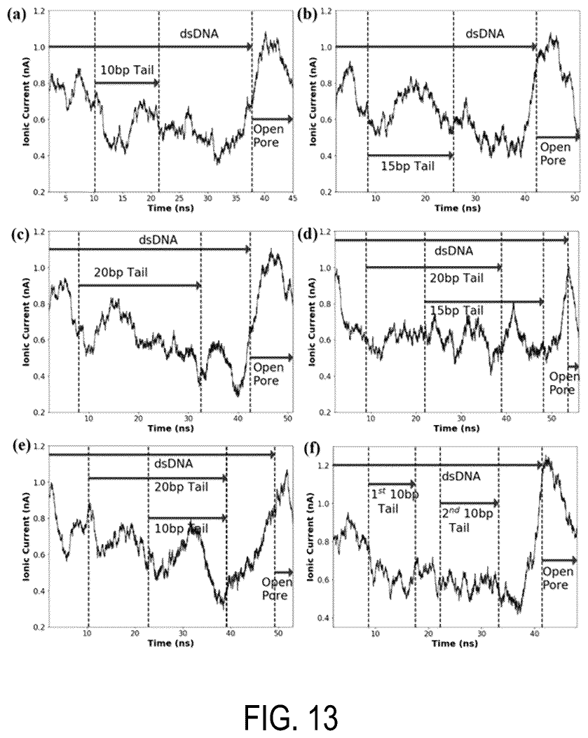

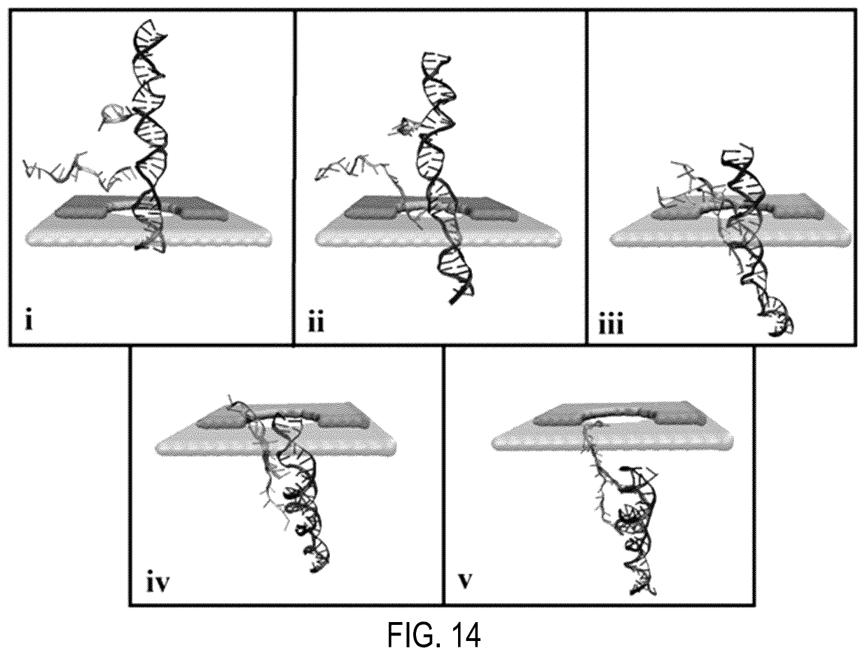

FIG. 13 depicts the use of ionic current through the pore for tail detection. The values of ionic current were small (between ˜0.3 nA and ˜1.2 nA) for three different levels (DNA alone in the pore, DNA with one tail in the pore, and DNA with two overlapping tails in the pore). There was an increase in the ionic current in some scenarios when tails were present inside the pore (˜18 ns in FIG. 14b, ˜15 ns in FIGS. 14c, and ˜34 ns in FIG. 14e), whereas for other scenarios, (˜15 ns in FIG. 14a, ˜12 ns in FIG. 14b, ˜10 ns and ˜20 ns to ˜30 ns in FIG. 14c, ˜12 ns, ˜28 ns and ˜38 ns in FIG. 14d, ˜12 ns and ˜25 ns in FIGS. 14d, and ˜14 ns, ˜17 ns, and ˜28 ns in FIG. 14e) the value decreased. This may be related to the use of low KCL concentration (0.1 M) solutions in order to improve the sensitivity of the transverse electronic conductance, which is related to the Debye length of the solution,

$${λ}_{d} = \frac{0.304}{\sqrt{I ( M )}} ( in nm ) ,$$

where I is the ionic molar concentration.

Transverse conductance signals provide better resolution compared to ionic current variations. Use of a 4-nm diameter nanopore increases conformational noise and can cause translocation anomalies, wherein the RNA tails stick on the upper surface of the MoS₂ membrane due to its hydrophobicity. As a result, the dsDNA can translocate through the pore first, even though the tails are placed far away from the end of the dsDNA. The snapshots of FIG. 14 illustrate this situation, which can cause erroneous signals in the transverse conductance. A 3 nm-diameter pore can restricts such movements, resulting in the entire payload biomolecule translocating uniformly through the pore, leading to increased ease of detecting tail dwell times.

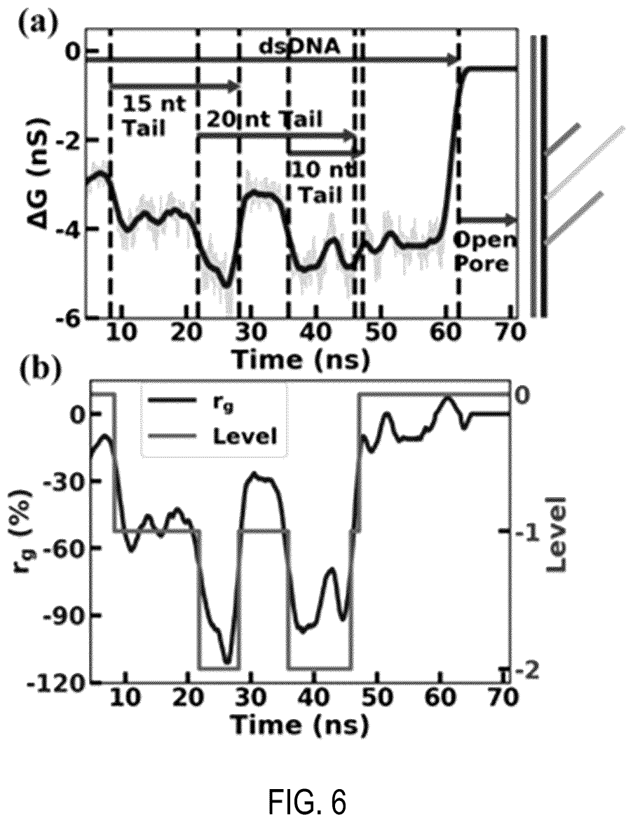

The techniques described herein can be extended to more than two overlapping tails translocating through the pore at the same time, and to longer backbone dsDNA strands. An 80 bp dsDNA background with three RNA tails was assessed—15 nt, 20 nt and 10 nt in sequence (placed 10 bp apart at 28^{th}, 38^{th} and 48^{th} bases, respectively). Plots for differential conductance and relative conductance, and level as this payload passed through a pore are shown in FIG. 6. For the piecewise linear model in the calculation of the relative conductance, it was assumed that the 80 bp DNA remained in its helicoidal shape (ΔG_{DNA-hel}) for the same duration (˜10 ns discussed in second regime of FIG. 2) as the 70 bp DNA. The level diagram of FIG. 6(b) shows that all the tail entries into and exits from the pore can successfully be determined.

The normalized dwell time, tₙₒᵣₘ, can be determined from the duration of levels by introducing an extra multiplier parameter, m, in Eqn. (2) to account for the decrease in DNA velocity, v_{dna}, during translocation caused by the increase in dsDNA length and mass. Here, the multiplier was set to 80/70, assuming that the velocity changed linearly with the length of the backbone DNA. The calculated t_{mod} values for 10 nt, 15 nt, and 20 nt tails were 6.57 ns, 13.47 ns, and 14.91 ns, respectively, and fell well within the range of the decision boundaries derived above. This demonstrates the robustness of the method described herein in accommodating various numbers of tails and dsDNA lengths. The effect of the multiplier, m, can vanish with the use of longer dsDNA strands as the ratio between two long DNA strands with a similar degree of length mismatch (˜10 bp) in lengths will be close to 1. This can also reduce the effect of the number of tails on the velocity of the whole structure, which has an effect on the normalized dwell times.

These simulation results establish that the techniques described herein can be used to detect the existence of both overlapping and non-overlapping RNA tails attached to a dsDNA structure by analyzing the transverse conductance variations of a solid-state MoS₂ nanopore. This can include distinguishing the lengths of the tails by the use of the normalized dwell times. Non-normalized dwell times showed a significant deviation due to changes in biomolecule velocity along the direction perpendicular to the membrane for overlapping vs. non-overlapping tails. Normalizing all the velocities with respect to the DNA velocity provided unique, unambiguous decision boundaries for each of the tails that were used to differentiate between the tail lengths. A separation of 10 bp between the tails worked better than a 15 bp separation, because in the latter case, there was an increase in velocity due to the uniform force being applied on both sides of the biomolecule, resulting in a shortened dwell time.

The techniques described herein can be extended to multiple tails and longer dsDNA strands. The techniques described herein can be implemented experimentally by first obtaining the dwell times and the velocity profile of known biomolecule payloads with tails, and then using the experimental data to establish decision boundaries based on normalized dwell times for detecting tails of unknown lengths. The difference between two neighboring tail lengths can be specified to be large enough to obtain unique decision boundaries for all of the tail lengths that may be present on a payload biomolecule. Separation distances between adjacent tails on the DNA can be chosen such that overlapping of more than two tails at a time (or some higher number of simultaneous tails for which the pore and measurement method have been adapted) does not take place inside the pore.

### Molecular Dynamics Simulations

All systems were built and analyzed using the Visual Molecular Dynamics (VMD) software. 9 nm×9 nm MoS₂ membranes were simulated using the coordinates of a standard 2D unit cell. For this membrane, Lennard-Jones parameters from Stewart, wt al. (Stewart, J. A.; Spearot, D. E. Atomistic Simulations of Nanoindentation on the Basal Plane of Crystalline Molybdenum Disulfide (MoS₂). Model. Simul. Mater. Sci. Eng. 2013, 21 (4), 045003) were used for all calculations, and all the atoms were fixed to their initial positions. Nanopores of a specified diameter were built by manually removing atoms from the desired region from the membrane. The dsDNA and the RNA tail structures were taken from the 3D-DART web server. CHARMM27 force fields were used to describe these biomolecules. To attach an RNA tail onto the backbone of a DNA, phosphate atoms and the phosphodiester bond from one of the nucleotides of the dsDNA were removed manually to create the simulated nick. Then, a bond was created between the 5′ carbon of this nucleotide and the phosphate group of one end of the RNA.

For each setup, the 2D membrane and the biomolecule were solvated in a water box with a simulated 0.1 M KCl solution. The MD simulations were performed using NAMD 2.13. Periodic boundary conditions were employed in all directions. The systems were maintained at 300 K using a Langevin thermostat. Time steps of 2 fs were used. A particle Mesh Ewald was used to evaluate long-range electrostatics. All systems were minimized for 5000 ps and then further equilibrated for 600 ps and 2 ns as an NPT and NVT ensemble, respectively. The trajectories of all atoms in the system were recorded at every 5000 steps until the DNA translocated entirely. These trajectory files were used to calculate the electrostatic potentials induced by the biomolecule around the semiconducting nanopore rim and its corresponding electronic transport as described below. An electric field was then applied to the system along the +z direction to drive the biomolecule through the nanopore. For all simulations, the electric field was set to 0.0417 V/nm to obtain and compare the dwell times of the tails.

### Electrostatic Potential Calculations

For each frame in the MD trajectory, the electrostatic potential, φ(r), was calculated numerically by the multi-grid method using the self-consistent Poisson Boltzmann's equation shown in (3), until convergence criterion was met:

$$∇ . [ ϵ ( r ) ∇ φ ( r ) ] = - e [ {C}_{{K}^{+}} ( r ) - {C}_{{Cl}^{-}} ( r ) ] - {ρ}_{DNA} ( r ) - {ρ}_{Tail ( s )} ( r ) , \qquad ( 3 )$$

where ρ_{DNA}(r) is the charge due to DNA, ϵ(r) is the local permittivity, C_{K}+(r) and C_{Cl}−(r) are the local electrolyte concentrations of K⁺ and Cl⁻ that obey the Poisson Boltzmann statistics given by the following equations.

$${C}_{{K}^{+}} ( r ) = {C}_{0} exp [ \frac{- e φ ( r )}{{k}_{B} T} ] \qquad ( 4 )$$

$${C}_{{Cl}^{-}} ( r ) = {C}_{0} exp [ \frac{e φ ( r )}{{k}_{B} T} ] \qquad ( 5 )$$

Here, C₀ is the nominal concentration of KCL in the solution, usually set to 0.1 M. The above two equations were solved numerically until convergence criteria were met.

### Electron Transport in MoS₂

The electronic transport in the MoS₂ membrane with a nanopore was treated with the semi-classical Boltzmann transport mechanism using Fermi's golden rule. The electrostatic potential variations due to the translocation of the biomolecules as well as the induced change of ion distribution were modeled by a perturbation in the form of a Dirac delta function to the transverse current. Hence, the conductance of the pore is given by

$${G}_{pore} = \frac{F}{{q}^{2} {ϕ}_{tot}^{2} {A}_{np}^{2}} \qquad ( 6 )$$

where qϕₜₒₜ is the total perturbation energy to the pore rim achieved in electrostatics calculation, Aₙₚ is the area of the nanopore, and F is a form factor determined by the MoS₂ ribbon width, carrier concentration, and pore position. The total conductance of a MoS₂ nanoribbon nanopore was calculated by the following Matthiessen's rule:

$$\frac{1}{{G}_{tot}} = \frac{1}{{G}_{ribbon}} + \frac{γ}{{G}_{pore}} \qquad ( 7 )$$

Here, G_{ribbon} is the conductance of the bare MoS₂ ribbon, and γ is the geometry aspect ratio of the nanoribbon.

### Ionic Current Calculation

The instantaneous ionic current/(t) through the nanopore was calculated for every trajectory frames using the following equation:

$$I ( t ) = \frac{1}{Δ t {L}_{z}} {\sum }_{i = 1}^{N} {q}_{i} ( {z}_{i} ( t + Δ t ) - {z}_{i} ( t ) )$$

where qᵢ and zᵢ are the charge and z coordinate of the i-th ion, respectively, Δt is the interval between successive trajectory frames, N is the total number of ions and L₂ is the z-coordinate dimension of the entire systems.

TABLE 1
Dwell times of RNA tails for different cases (a) without and
(b) with velocity normalization.

| (a) | | Single | With 10 nt | With 15 nt | With 20 nt |
|---|---|---|---|---|---|
| | 10 nt | 11.94 ns | — | — | 14.97 ns |
| | 15 nt | 17.1 ns | — | — | 23.43 ns |
| | 20 nt | 23.1 ns | 26.88 ns | 31.14 ns | — |
| (b) | | Single | With 10 nt | With 15 nt | With 20 nt |
| | 10 nt | 7.79 ns | — | — | 7.3 ns |
| | 15 nt | 11.96 ns | — | — | 12.28 ns |
| | 20 nt | 15.07 ns | 15.08 ns | 17.29 ns | — |

FIG. 2A is an illustration of aspects of a setup to detect an RNA tail (cyan ribbon) attached to a 70 bp dsDNA (blue and red ribbons). Simultaneous measurements of transverse currents and ionic currents were taken as the simulated biomolecule translocated through the nanopore.

FIG. 2B illustrates the differential conductance signal and number of DNA atoms near the pore for a 70 bp dsDNA. The plot includes instantaneous differential conductance (ΔG=G−Gₒₚₑₙ<0) (left axis, cyan curve) and number of atoms near the vicinity of the pore (right axis, red curve). Insets show that the DNA was deformed during the first part of translocation (bottom left corner) while it recovered the usual helicoidal shape at the later phase (right middle). Based on these changes, the instantaneous differential conductance was then modeled with a piecewise linear curve (ΔG_{DNA}) (left axis, solid blue curve) for further analysis.

FIG. 3 shows conductance signals for a 70 bp dsDNA with (a, b) a 20 nt RNA tail attached at 28^{th} base, (c, d) a 20 nt and a 15 nt RNA tails placed 10 bp apart at 28^{th} and 38^{th} base, respectively, and (e, f) a 20 nt and a 10 nt RNA tails placed 10 bp apart at 28^{th} and 38^{th} base, respectively. (a, c, e) show differential conductance (ΔG) as a function of time. The thick blue line represents the moving average with a 2.4 ns window while the gray line in the background is for the instantaneous signal. Different events during the translocation are denoted by red arrows and obtained from MD. Schematics of the respective biomolecule are shown beside the plot where the blue and red sticks denote the dsDNA, and the orange, cyan and the green sticks denote the 15 nt, the 20 nt and the 10 nt tails, respectively. (b, d, f) show relative conductance, r_{g} (left axis, blue curve) and calculated levels (right axis, red curve) for the respective biomolecules as a function of time. Here, level −1 represents the existence of a single non-overlapping tail, while level −2 represents the existence of two overlapping tails in the nanopore. Dwell time can be calculated using the duration of different levels.

FIG. 4 shows error bar plot of dwell times for different tails (a) before and (b) after normalization of velocity. Green dashed lines represent the decision boundaries in both plots. Normalized dwell times are more focused at the centers and eliminate overlap of dwell times between different regions.

FIG. 5 shows conductance signals and velocity for a dsDNA with two 10 nt RNA tails placed 15 bp apart at 28^{th} and 43^{rd} base, respectively. (a) shows differential conductance (ΔG) as a function of time. The thick blue line represents the moving average with a 2.4 ns window while the gray line in the background is for the instantaneous signal. Different events during the translocation are denoted by red arrows and obtained from MD. A schematic of the biomolecule is shown beside the plot where the blue and red sticks denote the dsDNA, and the green sticks denote the 10 nt tails. (b) shows relative conductance, r_{g} (left axis, blue curve) and calculated levels (right axis, red curve) for the same biomolecule as a function of time. Here, level −1 represents the existence of a single non-overlapping tail, and the durations of these levels are the dwell times for the tails. (d) shows DNA translocation velocity perpendicular to the nanopore membrane as a function of time during different stages of translocation.

FIG. 6 shows conductance signals for an 80 bp dsDNA with a 15 nt, 20 nt and a 10 nt RNA tails placed 10 bp apart at 28^{th}, 38^{th} and 48^{th} base, respectively. (a) shows differential conductance (ΔG) as a function of time. The thick blue line represents the moving average with a 2.4 ns window while the gray line is for the instantaneous signal. Different events during the translocation are denoted by red arrows and obtained from MD. A schematic of the biomolecule is shown beside the plot where the blue and red sticks denote the dsDNA, and the orange, cyan and the green sticks denote the 15 nt, the 20 nt and the 10 nt tails, respectively. (b) shows relative conductance, r_{g} (left axis, blue curve) and calculated levels (right axis, red curve) for the same biomolecule as a function of time. Here, level −1 represents the existence of a single non-overlapping tail, while level −2 represents the existence of two overlapping tails in the nanopore. Dwell time can be calculated using the duration of different levels.

FIG. 7 shows snapshots of key events during the translocation of a dsDNA with a 20 nt RNA tail through a 3 nm-diameter nanopore. (i) shows an initial part of the translocation. (ii) shows 20 nt RNA tail (cyan ribbon) entering the pore. (iii) shows the RNA tail leaving the pore. (iv) shows DNA (red and blue ribbons) exiting the pore.

FIG. 8 shows conductance signals for a 70 bp dsDNA with a 15 nt RNA tail attached at 28^{th} base. (a) shows differential conductance (ΔG) as a function of time. The thick blue line represents the moving average with a 2.4 ns window while the gray line in the background is for the instantaneous signal. Different events during the translocation are denoted by red arrows and obtained from MD. A schematic of the biomolecule is shown beside the plot where the blue and red sticks denote the dsDNA, and the orange stick denotes the 15 nt tail. (b) shows relative conductance, r_{g} (left axis, blue curve) and calculated levels (right axis, red curve) for the same biomolecule as a function of time. Here, level −1 represents the existence of a single non-overlapping tail, and the duration of this level is the dwell time for the tail.

FIG. 9 shows conductance signals for a 70 bp dsDNA with a 10 nt RNA tail attached at 28^{th} base. (a) shows differential conductance (ΔG) as a function of time. The thick blue line represents the moving average with a 2.4 ns window while the gray line in the background is for the instantaneous signal. Different events during the translocation are denoted by red arrows and obtained from MD. A schematic of the biomolecule is shown beside the plot where the blue and red sticks denote the dsDNA, and the green stick denotes the 10 nt tail. (b) shows relative conductance, r_{g} (left axis, blue curve) and calculated levels (right axis, red curve) for the same biomolecule as a function of time. Here, level −1 represents the existence of a single non-overlapping tail, and the duration of this level is the dwell time for the tail.

FIG. 10 shows snapshots of key events during the translocation of a 70 bp dsDNA with a 20 nt and a 15 nt RNA tails placed 10 bp apart from each other. (i) shows an initial part of the translocation. (ii) shows the 20 nt RNA tail (cyan ribbon) entering the pore. (iii) shows the 15 nt RNA tail (orange ribbon) entering the pore. (iv) shows the 20 nt RNA tail leaving the pore. (v) shows the 15 nt RNA tail leaving the pore. (vi) shows DNA (red and blue ribbons) exiting the pore.

FIG. 11 shows snapshots of key events during the translocation of a 70 bp dsDNA with a 20 nt and a 10 nt RNA tails placed 10 bp apart from each other through a 3-nm diameter pore. (i) shows an initial part of the translocation. (ii) shows the 20 nt RNA tail (cyan ribbon) entering the pore. (iii) shows the 10 nt RNA tail (green ribbon) entering the pore. (iv) shows the 20 nt and 10 nt RNA tails almost simultaneously leaving the pore. (v) shows DNA (red and blue ribbons) exiting the pore.

FIG. 12 shows the velocity of a DNA molecule with two RNA tails along the vertical direction of the nanopore membrane as a function of time during different stages of translocation. Black dashed vertical lines show the boundaries of different events.

FIG. 13 shows ionic current traces for different cases for translocation of a dsDNA with RNA tails through a 3-nm diameter nanopore. Ionic current as a function of time for a dsDNA with a single tail ((a) 10 nt, (b) 15 nt and (c) 20 nt), two tails with 10 nt separation ((d) 20 nt and 15 nt, (e) 20 nt and 10 nt) and (f) two 10 nt tails with 15 bp separation. The thick blue line represents the moving average with a 5 ns window. Different events during the translocation are denoted by red arrows and obtained from MD.

FIG. 14 shows snapshots of key events during the translocation of a 70 bp dsDNA with a 20 nt and a 10 nt RNA tails placed 10 bp apart from each other through a 3-nm diameter pore. (i) shows an initial part of the translocation. (ii) shows the 20 nt RNA tail (cyan ribbon) entering the pore. (iii) shows the 10 nt RNA tail (green ribbon) entering the pore. (iv) shows DNA (red and blue ribbons) exiting the pore. (v) shows the 20 nt and 10 nt RNA tails almost simultaneously leaving the pore. (iii) and (iv) show the sticking of the tails on the upper surface of the membrane while the dsDNA goes through the pore.

### III. EXAMPLE SYSTEMS

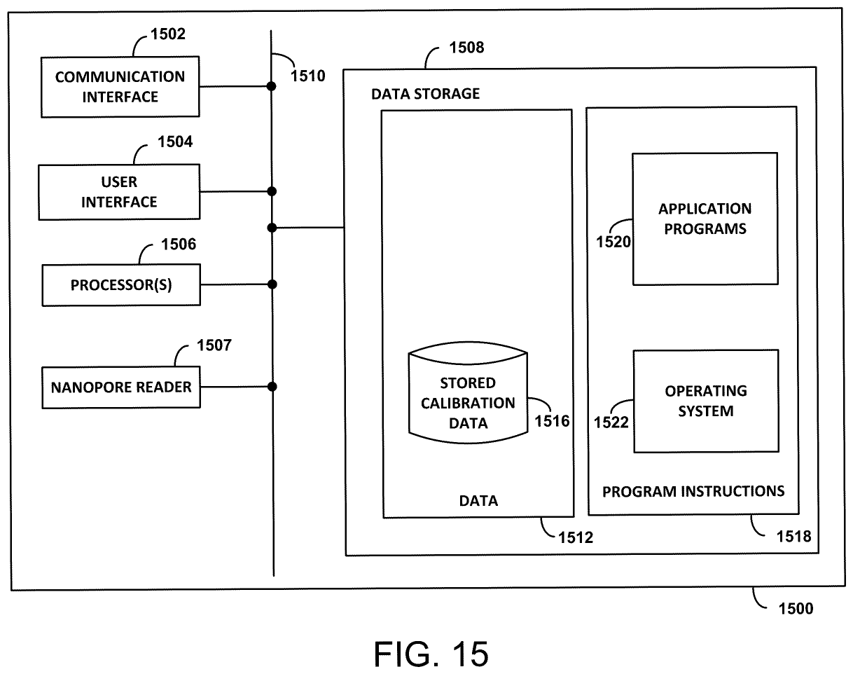

FIG. 15 illustrates an example system 1500 that may be used to implement the methods and/or apparatus described herein. By way of example and without limitation, system 1500 may be or include a computer (such as a desktop, notebook, tablet, handheld computer, and/or a server), elements of a laboratory instrument (e.g., a system configured to be installed and used in a laboratory context to perform the processes or methods described herein), elements of a data storage device (e.g., a device configured to read data stored in payload biomolecules from stored samples of such biomolecules), or some other type of device or system or combination of devices and/or systems. It should be understood that elements of system 1500 may represent a physical instrument and/or computing device such as a server, a particular physical hardware platform on which applications operate in software, or other combinations of hardware and software that are configured to carry out functions as described herein.

As shown in FIG. 15, system 1500 may include a communication interface 1502, a user interface 1504, one or more processors 1506, one or more nanopore readers 1507, and data storage 1508, all of which may be communicatively linked together by a system bus, network, or other connection mechanism 1510.

Communication interface 1502 may function to allow system 1500 to communicate, using analog or digital modulation of electric, magnetic, electromagnetic, optical, or other signals, with other devices (e.g., with systems that can receive data read from patterns of tails of payload biomolecules in order to, e.g., restore the contents of a database from a long-term information storage implemented in such payload biomolecules), access networks, and/or transport networks. Thus, communication interface 1502 may facilitate circuit-switched and/or packet-switched communication, such as plain old telephone service (POTS) communication and/or Internet protocol (IP) or other packetized communication. For instance, communication interface 1502 may include a chipset and antenna arranged for wireless communication with a radio access network or an access point. Also, communication interface 1502 may take the form of or include a wireline interface, such as an Ethernet, Universal Serial Bus (USB), or High-Definition Multimedia Interface (HDMI) port. Communication interface 1502 may also take the form of or include a wireless interface, such as a WiFi, BLUETOOTH®, global positioning system (GPS), or wide-area wireless interface (e.g., WiMAX, 3GPP Long-Term Evolution (LTE), or 3GPP 5G). However, other forms of physical layer interfaces and other types of standard or proprietary communication protocols may be used over communication interface 1502. Furthermore, communication interface 1502 may comprise multiple physical communication interfaces (e.g., a WiFi interface, a BLUETOOTH® interface, and a wide-area wireless interface).

User interface 1504 may function to allow system 1500 to interact with a user, for example to receive input from and/or to provide output to the user. Thus, user interface 1504 may include input components such as a keypad, keyboard, touch-sensitive or presence-sensitive panel, computer mouse, trackball, joystick, microphone, and so on. User interface 1504 may also include one or more output components such as a display screen which, for example, may be combined with a presence-sensitive panel. The display screen may be based on CRT, LCD, and/or LED technologies, or other technologies now known or later developed. User interface 1504 may also be configured to generate audible output(s), via a speaker, speaker jack, audio output port, audio output device, earphones, and/or other similar devices. The user interface 1504 may be operable to permit a user to initiate a calibration procedure to, e.g., specify that payload biomolecules being read by the nanopore reader 1507 correspond to a specified calibration structure and thus that calibration data relating to time-varying correction factors, velocity normalization factors, or other calibration data for the system (e.g., for one or more pores of the nanopore reader 1507) should be determined from current outputs of the nanopore reader 1507. The user interface 1504 may be operable to permit a user to initiate some other operations of the system 1500, e.g., to indicate that a payload-containing solution has been inserted into the system 1500 (e.g., into a sample container thereof) and thus that the nanopore reader 1507 can now be operated to read out the information encoded in the payload molecule(s) of the payload-containing solution.

Processor(s) 1506 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). Data storage 1508 may include one or more volatile and/or non-volatile storage components, such as magnetic, optical, flash, or organic storage, and may be integrated in whole or in part with processor(s) 1506 and/or with some other element of the system. Data storage 1508 may include removable and/or non-removable components.

Processor(s) 1506 may be capable of executing program instructions 1518 (e.g., compiled or non-compiled program logic and/or machine code) stored in data storage 1508 to carry out the various functions described herein. Therefore, data storage 1508 may include a non-transitory computer-readable medium, having stored thereon program instructions that, upon execution by system 1500, cause system 1500 to carry out any of the methods, processes, or functions disclosed in this specification and/or the accompanying drawings. The execution of program instructions 1518 by processor(s) 1506 may result in processor 1506 using data 1512.

By way of example, program instructions 1518 may include an operating system 1522 (e.g., an operating system kernel, device driver(s), and/or other modules) and one or more application programs 1520 (e.g., functions for executing the methods described herein) installed on system 1500. Data 1512 may include stored calibration data 1516 (e.g., stored sets of time-varying conductance correction factors, velocity normalization factors, or other information about the characteristics of one or more pores of the nanopore reader 1507) that can be used to determine how to operate the nanopore reader 1507 to read out information in payload biomolecule(s) presented to the system 1500.

Application programs 1520 may communicate with operating system 1522 through one or more application programming interfaces (APIs). These APIs may facilitate, for instance, application programs 1520 transmitting or receiving information via communication interface 1502, receiving and/or displaying information on user interface 1504, operating the nanopore reader 1507, and so on.

Application programs 1520 may take the form of “apps” that could be downloadable to system 1500 through one or more online application stores or application markets (via, e.g., the communication interface 1502). However, application programs can also be installed on system 1500 in other ways, such as via a web browser or through a physical interface (e.g., a USB port) of the system 1500.

Nanopore reader 1507 may include voltage generators, amplifiers, switches, controlled-current and/or controlled-voltage sources, clocks, analog-to-digital converters, or other elements to controllably drive payload biomolecules through pore(s) of the nanopore reader 1507, to detect ionic currents through such pores and/or transverse electronic conductances of barriers that include such pores as the payload biomolecules transit through the pore(s), and/or to perform other operations related to reading out information from one or more payload biomolecules as described herein. The nanopore reader 1507 could include multiple pores (e.g., formed in a single barrier, or formed in respective different barriers) to facilitate simultaneously reading out information from multiple different payload biomolecules. Additionally or alternatively, the system 1500 could include multiple nanopore readers 1507 to facilitate simultaneous readout from multiple payload biomolecules. The system 1500 could include pumps, valves, or other fluidic or microfluidic elements to facilitate directing payload-bearing solution to the nanopore reader 1507 (e.g., to specified pore(s) thereof).

### IV. EXAMPLE METHODS

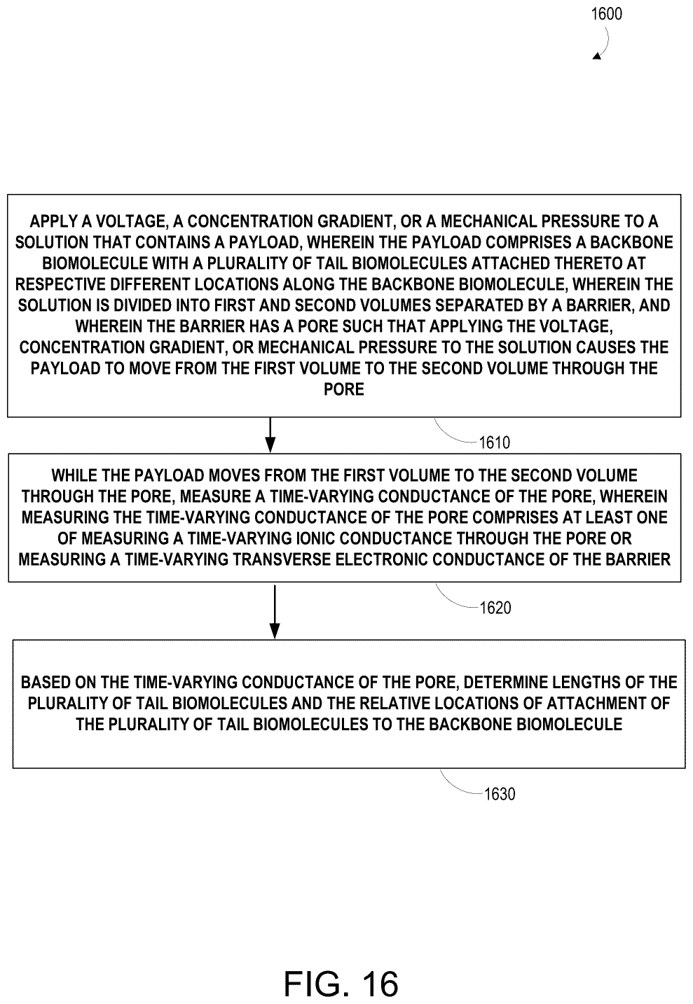

FIG. 16 depicts an example method 1600. The method 1600 includes applying a voltage, a concentration gradient, or a mechanical pressure to a solution that contains a payload, wherein the payload comprises a backbone biomolecule with a plurality of tail biomolecules attached thereto at respective different locations along the backbone biomolecule, wherein the solution is divided into first and second volumes separated by a barrier, and wherein the barrier has a pore such that applying the voltage, concentration gradient, or mechanical pressure to the solution causes the payload to move from the first volume to the second volume through the pore (1610). The method 1600 additionally includes, while the payload moves from the first volume to the second volume through the pore, measuring a time-varying conductance of the pore, wherein measuring the time-varying conductance of the pore comprises at least one of measuring a time-varying ionic conductance through the pore or measuring a time-varying transverse electronic conductance of the barrier (1620). The method 1600 additionally includes, based on the time-varying conductance of the pore, determining lengths of the plurality of tail biomolecules and the relative locations of attachment of the plurality of tail biomolecules to the backbone biomolecule (1630). The method 1600 could include additional steps or features.

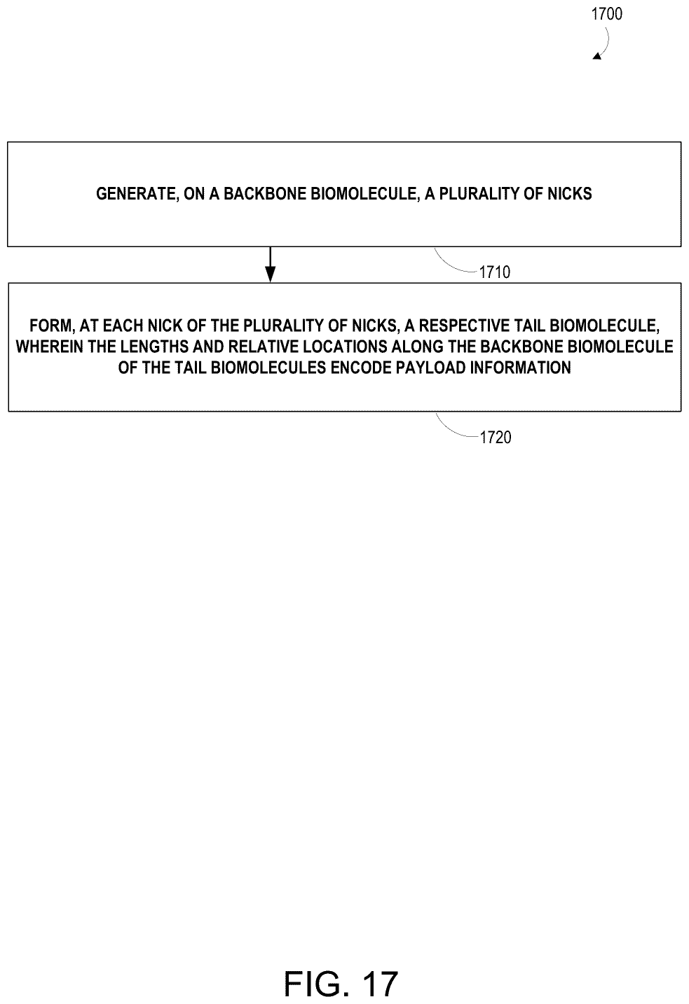

FIG. 17 depicts an example method 1700. The method 1700 includes generating, on a backbone biomolecule, a plurality of nicks (1710). The method 1700 additionally includes forming, at each nick of the plurality of nicks, a respective tail biomolecule, wherein the lengths and relative locations along the backbone biomolecule of the tail biomolecules encode payload information (1720). The method 1700 could include additional steps or features.

It should be understood that arrangements described herein are for purposes of example only. As such, those skilled in the art will appreciate that other arrangements and other elements (e.g. machines, interfaces, operations, orders, and groupings of operations, etc.) can be used instead of or in addition to the illustrated elements or arrangements.

### V. CONCLUSION

It should be understood that arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

We claim:

1. A method, comprising:
applying a voltage, a concentration gradient, or a mechanical pressure to a solution that contains a payload, wherein the payload comprises a backbone biomolecule with a plurality of tail biomolecules attached thereto at respective different locations along the backbone biomolecule, wherein the solution is divided into first and second volumes separated by a barrier, and wherein the barrier has a pore such that applying the voltage, concentration gradient, or mechanical pressure to the solution causes the payload to move from the first volume to the second volume through the pore;
while the payload moves from the first volume to the second volume through the pore, measuring a time-varying conductance of the pore, wherein measuring the time-varying conductance of the pore comprises at least one of measuring a time-varying ionic conductance through the pore or measuring a time-varying transverse electronic conductance of the barrier; and
based on the time-varying conductance of the pore, determining lengths of the plurality of tail biomolecules and the relative locations of attachment of the plurality of tail biomolecules to the backbone biomolecule.

2. The method of claim 1, wherein the lengths of the plurality of tail biomolecules and their relative locations of attachment to the backbone biomolecule are such that, during at least one period of time while the payload moves from the first volume to the second volume through the pore, at least two of the plurality of tail biomolecules move through the pore simultaneously.

3. The method of claim 2, wherein the lengths of the plurality of tail biomolecules and their relative locations of attachment to the backbone biomolecule are such that, while the payload moves from the first volume to the second volume through the pore, no more than two of the plurality of tail biomolecules move through the pore simultaneously, wherein the pore has a diameter that is tuned to a size of the payload, wherein the backbone biomolecule is a double strand of deoxyribonucleic acid, wherein the tail biomolecules are ribonucleic acid tails, and wherein the pore has a diameter between 2.8 and 3.2 nanometers.

4. The method of claim 1, wherein determining the lengths of the plurality of tail biomolecules and the relative locations of attachment of the plurality of tail biomolecules to the backbone biomolecule comprises subtracting, from the time-varying conductance of the pore, a time-varying correction factor that is dependent on a length of the backbone biomolecule.

5. The method of claim 4, wherein the time-varying correction factor is a piecewise linear function having an initial constant segment corresponding to an initial portion of the backbone biomolecule, a second constant segment corresponding to an end portion of the backbone biomolecule, and a terminal constant segment corresponding to the backbone biomolecule having exited the pore after moving through the pore, wherein a first corrective conductance value of the initial constant segment is greater than a second corrective conductance value of the second constant segment, and wherein a corrective conductance value of the terminal constant segment is greater than the corrective conductance value of the initial constant segment.

6. The method of claim 4, wherein determining the lengths of the plurality of tail biomolecules and the relative locations of attachment of the plurality of tail biomolecules to the backbone biomolecule comprises:
normalizing the time-varying conductance of the pore, and
comparing the time-varying conductance of the pore as normalized to a set of one or more thresholds to determine a time-varying number of the plurality of tail biomolecules moving through the pore.

7. The method of claim 6, wherein determining the lengths of the plurality of tail biomolecules and the relative locations of attachment of the plurality of tail biomolecules to the backbone biomolecule comprises, based on the time-varying number of the plurality of tail biomolecules that are moving through the pore, reconstructing the locations, along the length of the backbone biomolecule, of both ends of each tail biomolecule of the plurality of tail biomolecules when aligned against the backbone biomolecule.

8. The method of claim 7, wherein determining a length of a particular tail biomolecule of the plurality of tail biomolecules comprises:
determining a duration of time that the particular tail biomolecule took to move through the pore, and
normalizing the duration of time based on a relationship between velocity with which the payload moves through the pore on a number of tail biomolecules of the payload that are moving through the pore.

9. The method of claim 8, wherein normalizing the duration of time based on the relationship between the velocity with which the payload moves through the pore on the number of tail biomolecules of the payload that are moving through the pore comprises determining a sum of at least:
a first duration of the time that the particular tail biomolecule took to move through the pore while no other tail biomolecule of the plurality of tail biomolecules moved through the pore, normalized by a first velocity factor, and
a second duration of time that the particular tail biomolecule took to move through the pore while at least one and at most one other tail biomolecule of the plurality of tail biomolecules moved through the pore, normalized by a second velocity factor that differs from the first velocity factor.

10. The method of claim 9, wherein the time-varying correction factor is a piecewise linear function having an initial constant segment corresponding to an initial portion of the backbone biomolecule, a second constant segment corresponding to an end portion of the backbone biomolecule, and a terminal constant segment corresponding to the backbone biomolecule having exited the pore after moving through the pore, wherein a corrective conductance value of the initial constant segment is greater than a corrective conductance value of the second constant segment, and wherein a corrective conductance value of the terminal constant segment is greater than the corrective conductance value of the initial constant segment.

11. The method of claim 1, wherein the plurality of tail biomolecules have respective lengths and wherein a minimum spacing between neighboring locations of attachment of the plurality of tail biomolecules to the backbone biomolecule is greater than half a maximum length of the plurality of tail biomolecules.

12. The method of claim 1, wherein the lengths of the plurality of tail biomolecules and the relative locations of attachment of the plurality of tail biomolecules to the backbone biomolecule encode payload information, and wherein the method further comprises recovering the payload information based on the lengths of the plurality of tail biomolecules and the relative locations of attachment of the plurality of tail biomolecules to the backbone biomolecule.

13. A non-transitory computer readable medium having stored thereon program instructions executable by at least one processor to cause the at least one processor to perform a method comprising:
applying a voltage, a concentration gradient, or a mechanical pressure to a solution that contains a payload, wherein the payload comprises a backbone biomolecule with a plurality of tail biomolecules attached thereto at respective different locations along the backbone biomolecule, wherein the solution is divided into first and second volumes separated by a barrier, and wherein the barrier has a pore such that applying the voltage, concentration gradient, or mechanical pressure to the solution causes the payload to move from the first volume to the second volume through the pore;
while the payload moves from the first volume to the second volume through the pore, measuring a time-varying conductance of the pore, wherein measuring the time-varying conductance of the pore comprises at least one of measuring a time-varying ionic conductance through the pore or measuring a time-varying transverse electronic conductance of the barrier; and
based on the time-varying conductance of the pore, determining lengths of the plurality of tail biomolecules and the relative locations of attachment of the plurality of tail biomolecules to the backbone biomolecule A

14. The non-transitory computer readable medium of claim 13, wherein determining the lengths of the plurality of tail biomolecules and the relative locations of attachment of the plurality of tail biomolecules to the backbone biomolecule comprises subtracting, from the time-varying conductance of the pore, a time-varying correction factor that is dependent on a length of the backbone biomolecule.

15. The non-transitory computer readable medium of claim 14, wherein determining the lengths of the plurality of tail biomolecules and the relative locations of attachment of the plurality of tail biomolecules to the backbone biomolecule comprises:
normalizing the time-varying conductance of the pore,
comparing the time-varying conductance of the pore as normalized to a set of one or more thresholds to determine a time-varying number of the plurality of tail biomolecules moving through the pore, and
based on the time-varying number of the plurality of tail biomolecules that are moving through the pore, reconstructing the locations, along the length of the backbone biomolecule, of both ends of each tail biomolecule of the plurality of tail biomolecules when aligned against the backbone biomolecule.

16. The non-transitory computer readable medium of claim 15, wherein determining a length of a particular tail biomolecule of the plurality of tail biomolecules comprises:
determining a duration of time that the particular tail biomolecule took to move through the pore, and
normalizing the duration of time based on a relationship between velocity with which the payload moves through the pore on a number of tail biomolecules of the payload that are moving through the pore, wherein normalizing the duration of time comprises determining a sum of at least:
a first duration of the time that the particular tail biomolecule took to move through the pore while no other tail biomolecule of the plurality of tail biomolecules moved through the pore, normalized by a first velocity factor, and
a second duration of time that the particular tail biomolecule took to move through the pore while at least one and at most one other tail biomolecule of the plurality of tail biomolecules moved through the pore, normalized by a second velocity factor that differs from the first velocity factor.

17. A system comprising:
a barrier having a pore; and
a controller comprising one or more processors, wherein the controller is configured to perform controller operations comprising:
applying a voltage, a concentration gradient, or a mechanical pressure to a solution that contains a payload, wherein the payload comprises a backbone biomolecule with a plurality of tail biomolecules attached thereto at respective different locations along the backbone biomolecule, wherein the solution is divided into first and second volumes by the barrier, and wherein applying the voltage, concentration gradient, or mechanical pressure to the solution causes the payload to move from the first volume to the second volume through the pore;
while the payload moves from the first volume to the second volume through the pore, measuring a time-varying conductance of the pore, wherein measuring the time-varying conductance of the pore comprises at least one of measuring a time-varying ionic conductance through the pore or measuring a time-varying transverse electronic conductance of the barrier; and
based on the time-varying conductance of the pore, determining lengths of the plurality of tail biomolecules and the relative locations of attachment of the plurality of tail biomolecules to the backbone biomolecule.

18. The system of claim 17, wherein the lengths of the plurality of tail biomolecules and their relative locations of attachment to the backbone biomolecule are such that, while the payload moves from the first volume to the second volume through the pore, no more than two of the plurality of tail biomolecules move through the pore simultaneously, wherein the pore has a diameter that is tuned to a size of the payload, wherein the backbone biomolecule is a double strand of deoxyribonucleic acid, wherein the tail biomolecules are ribonucleic acid tails, and wherein the pore has a diameter between 2.8 and 3.2 nanometers.

19. The system of claim 17, wherein determining the lengths of the plurality of tail biomolecules and the relative locations of attachment of the plurality of tail biomolecules to the backbone biomolecule comprises subtracting, from the time-varying conductance of the pore, a time-varying correction factor that is dependent on a length of the backbone biomolecule.

20. The system of claim 19, wherein determining the lengths of the plurality of tail biomolecules and the relative locations of attachment of the plurality of tail biomolecules to the backbone biomolecule comprises:
normalizing the time-varying conductance of the pore,
comparing the time-varying conductance of the pore as normalized to a set of one or more thresholds to determine a time-varying number of the plurality of tail biomolecules moving through the pore, and
based on the time-varying number of the plurality of tail biomolecules that are moving through the pore, reconstructing the locations, along the length of the backbone biomolecule, of both ends of each tail biomolecule of the plurality of tail biomolecules when aligned against the backbone biomolecule.
